SEQUENCE LISTING (9 sequences)

SEQ ID NO: 1 - protein, 16 aa, Homo sapiens
LYEGRNPGFYVEANPM

SEQ ID NO: 2 - protein, 15 aa, Homo sapiens
PEETLVIALYDQTND

SEQ ID NO: 3 - protein, 14 aa, Homo sapiens
EQEDEPEGIYGVLF

SEQ ID NO: 4 - protein, 14 aa, Homo sapiens
EQEDEPEGISGVLF

SEQ ID NO: 5 - protein, 7 aa, Homo sapiens
YIYGSFK

SEQ ID NO: 6 - protein, 5 aa, Homo sapiens
GGGGS

SEQ ID NO: 7 - protein, 7 aa, Homo sapiens
KFSGYIY

SEQ ID NO: 8 - protein, 108 aa, Homo sapiens
NNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCG

SEQ ID NO: 9 - protein, 101 aa, Homo sapiens
QTNAHESKEWYHASLTRAQAEHMLMRVPRDGAFLVRKRNEPNSYAISFRAEGKIKHCRVQQEGQEGQTVMLGNSEFDSLVDLISYYEKHPLYRKMKLRYPI

(19) United States
(12) Patent Application Publication
(10) Pub. No.: US 2008/0247953 A1
(43) Pub. Date: Oct. 9, 2008

(54) METHODS AND COMPOSITIONS FOR AFFECTING SUBSTRATE DOCKING OF KINASES IN MODULATING CELLULAR ACTIVITIES

(75) Inventors: Amy H. Andreotti, Ames, IA (US); Raji E. Joseph, Ames, IA (US); Lie Min, Ames, IA (US)

Correspondence Address: MCKEE, VOORHEES & SEASE, P.L.C., 801 GRAND AVENUE, SUITE 3200, DES MOINES, IA, 50309-2721, US

(73) Assignee: IOWA STATE UNIVERSITY RESEARCH FOUNDATION, INC., Ames, IA (US)

(21) Appl. No.: 11/697,328

(22) Filed: Apr. 6, 2007

(51) Int. Cl. A61K 49/00; A61K 31/70; A61K 38/02; A61P 27/00; A61P 3/10; A61P 37/00; C12Q 1/48; G01N 33/53; A61P 9/10; A61P 35/00; A61P 29/00; A61P 17/06; A61K 39/395

(52) U.S. Cl. 424/9.2; 424/130.1; 435/15; 435/7.21; 514/2; 514/23; 514/43

(57) ABSTRACT

The present invention relates to drug screening assays, therapeutic protocols and pharmaceutical compositions designed to target non-receptor tyrosine family kinases and components of the tyrosine kinase family signal transduction pathways. This includes primarily diseases or conditions associated with immune responses and can include treatment for cancers as well as various immune disorders. The invention reports a novel substrate SH2 domain docking mechanism apart from the kinase active site that is required for appropriate tyrosine phosphorylation by these tyrosine kinases.

### GRANT REFERENCE

This work was funded in part by NIH grant no. R01 AI043957. The government may have certain rights in this invention.

### FIELD OF THE INVENTION

The present invention relates to drug screening assays, therapeutic protocols and pharmaceutical compositions designed to target the non-receptor protein tyrosine kinase family and components of the tyrosine kinase signal transduction pathways. The invention reports a novel substrate docking mechanism required for appropriate immune cell activation by these tyrosine kinases that forms the basis of chemical intervention techniques.

### BACKGROUND OF THE INVENTION

Protein kinases (PKs) are enzymes that catalyze the phosphorylation of hydroxyl groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non life-threatening diseases such as psoriasis to extremely virulent diseases such as glioblastoma (brain cancer). PKs phosphorylate the hydroxyl group on tyrosine (the protein tyrosine kinases (PTKs)) or the hydroxyl group on serine or threonine (the serine-threonine kinases (STKs)).

Certain growth factor receptors exhibiting PK activity are known as receptor tyrosine kinases (RTKs). They comprise a large family of transmembrane receptors with diverse biological activity. At present, at least nineteen (19) distinct subfamilies of RTKs have been identified. In addition to the RTKs, there also exists a family of intracellular PTKs called “non-receptor tyrosine kinases” or “cellular tyrosine kinases.” This latter designation, abbreviated “CTK”, will be used herein. CTKs do not contain extracellular and transmembrane domains. This group, at present, includes 32 proteins in 9 different subgroups. These proteins respond to extracellular stimuli by means of modular units like Src homology 2 (SH2), Src homology 3 (SH3), and Pleckstrin homology (PH) domains, or modification by lipids (e.g. Myristate), for appropriate subcellular localization. The family includes the Abl, Csk, FAK, JAK, Src, Syk, and Tec sub families. The Src subfamily is the largest and includes Src, Fyn, Hck, Lyn, Yes, Yrk, Blk, Fgr, and Lck. Src was first identified as the transforming protein in Rous sarcoma virus. The second largest family of non-receptor protein tyrosine kinases is the Tec family and includes Itk, Btk, Tec, Bmx, and Rlk. These kinases are expressed in hematopoietic cells.

Most of the proteins of each of these families of non-receptor PTKs couple to cellular receptors that lack enzymatic activity themselves. This class of receptors includes all of the cytokine receptors (e.g. the interleukin-2 (IL-2) receptor) as well as the CD4 and CD8 cell surface glycoproteins of T cells, and the T cell and B cell antigen receptors (TCR and BCR).

RTKs, and CTKs have all been implicated in a host of pathogenic conditions including significantly, cancer. Other pathogenic conditions, which have been associated with tyrosine kinases include, without limitation, psoriasis, hepatic cirrhosis, diabetes, atherosclerosis, angiogenesis, restenosis, ocular diseases, rheumatoid arthritis and other inflammatory disorders, autoimmune diseases and a variety of renal disorders.

As can be seen there is a continuing need to understand and develop therapeutic interventions relating to CTKs and their regulation of cellular activities.

### BRIEF SUMMARY OF THE INVENTION

The present invention relates to the discovery of a novel substrate docking mechanism for the non-receptor protein tyrosine kinases. An SH2 domain within the substrates of Src and Tec family kinases serves as a docking module for recognition and phosphorylation by the kinase. In each of the substrate-kinase pairs examined, the SH2 domain within the substrate is remote from the site of phosphorylation and shuttles the substrate to the appropriate kinase domain by enhancing the affinity of the enzyme/substrate complex. Corresponding substrates that lack the SH2 domain are not phosphorylated by the kinase. In contrast to the canonical, phosphotyrosine dependent binding behavior of SH2 domains, we show that the SH2 domains of the kinase substrates perform this docking function in a phosphotyrosine-independent manner. The direct interaction between substrate SH2 domain and the kinase domain of the enzyme takes place outside of the active site of the kinase domain. This remote docking mechanism thus allows for strategies to interrupt CTK signaling in a manner that displaces substrate from the kinase domain rather than the direct inhibition of a kinase active site. The present invention thus relates to methods to increase CTK signaling and CTK substrate interactions by addition of additional docking sites to substrates, as well as methods to decrease CTK signaling by providing additional substrate docking sites that are not bound to substrates thus competitively inhibiting the CTK substrate interaction. The invention also relates to drug screening protocols designed to identify agents which can modulate this interaction and thus provides a novel target for therapeutic and/or prophylactic intervention by small molecules, antibodies, and the like for treatment of diseases such as cancers, psoriasis, hepatic cirrhosis, diabetes, atherosclerosis, angiogenesis, restenosis, ocular diseases, rheumatoid arthritis and other inflammatory disorders, autoimmune diseases and a variety of renal disorders.

The invention is illustrated by way of working examples that demonstrate that phosphorylation mediated by CTK family members requires a protein-protein interaction between the kinase enzyme and an SH2 domain of the substrate. This novel docking mechanism mediates activation of a CTK signaling cascade and is therefore an essential function required for regulation of cellular activities including cellular proliferation and immune cell activation. There are two unexpected findings in this invention. The first is the phosphotyrosine-independent nature of the SH2 mediated docking interaction and the second is the remote nature of the docking interaction; substrate SH2 domain binds to the kinase domain of the enzyme in a manner that has no direct effect on the kinase active site. These findings indicate that two previously undescribed protein surfaces can be targeted to disrupt substrate phosphorylation by the CTKs. Thus, this present invention relates to a method of modulating the catalytic activity of CTKs in a mammal in need thereof by contacting the docking site (on either the substrate SH2 domain and/or the enzyme kinase domain) with an inhibitory compound that disrupts the interaction of the two, or by stimulating the interactions by taking advantage of this docking activity by, for example providing additional SH2 domains to the cells.

As used herein, the term “modulation” or “modulating” refers to the alteration of the catalytic activity of cellular tyrosine kinases (CTKs).

The term “catalytic activity” as used herein refers to the rate of phosphorylation of tyrosine under the influence, direct or indirect, of CTKs.

The term “contacting” as used herein refers to bringing a compound and a target CTK or CTK substrate together in such a manner that the compound can affect the catalytic activity of the CTK away from the kinase active site, either directly; i.e., by interacting with the kinase itself, or indirectly; i.e., by interacting with another molecule on which the catalytic activity of the kinase is dependent. Such “contacting” can be accomplished “in vitro,” i.e., in a test tube, a petri dish or the like. In a test tube, contacting may involve only a compound and a CTK or substrate of interest or it may involve whole cells. Cells may also be maintained or grown in cell culture dishes and contacted with a compound in that environment. In this context, the ability of a particular compound to affect a CTK related disorder, i.e., the IC₅₀ of the compound, can be determined before use of the compounds in vivo with more complex living organisms is attempted. For cells outside the organism, multiple methods exist, and are well known to those skilled in the art, to get the CTKs in contact with the compounds including, but not limited to, direct cell microinjection and numerous transmembrane carrier techniques.

The above-referenced CTK whose catalytic activity is modulated may be selected from the group comprising the Abl, Csk, FAK, JAK, Src, Syk, and Tec sub families. In a preferred embodiment the CTK is a member of the Tec family and is selected from the group consisting of Itk, Btk, Tec, Bmx, and Rlk. In yet another preferred embodiment the CTK is a member of the Src subfamily is selected from the group consisting of Src, Fyn, Hck, Lyn, Yes, Yrk, Blk, Fgr and Lck.

In another aspect, this invention relates to a method for treating or preventing a CTK-related disorder in a mammal in need of such treatment comprising administering to the mammal a therapeutically effective amount of one or more of the modulating compounds described above.

As used herein, “CTK-related disorder,” CTK driven disorder,” and “abnormal CTK activity” all refer to a condition characterized by inappropriate (i.e., diminished or, more commonly, excessive) CTK catalytic activity, where the particular CTK can be member of the Abl, Csk, FAK, JAK, Src, Syk, and/or Tec sub families. Inappropriate catalytic activity can arise as the result of either: (1) CTK expression in cells which normally do not express CTKs; (2) increased CTK expression leading to unwanted cell proliferation, differentiation and/or growth; (3) decreased CTK expression leading to unwanted reductions in cell proliferation, differentiation and/or growth; or, (4) CTK mutation(s) leading to unregulated catalytic activity either in the positive or negative sense. Excessive-activity of a CTK refers to either amplification of the gene encoding a particular CTK or its substrate, or production of a level of CTK activity which can correlate with a cell proliferation, differentiation and/or growth disorder (that is, as the level of the CTK increases, the severity of one or more symptoms of a cellular disorder increase as the level of the CTK activity decreases). In addition, affecting normal levels of CTK activity by use of the modulating compounds described above is envisioned to achieve suppression of cell signaling (for example immunosuppresion following organ transplantation).

“Treat,” “treating” or “treatment” with regard to a CTK-related disorder refers to alleviating or abrogating the cause and/or the effects of a CTK-related disorder.

As used herein, the terms “prevent”, “preventing” and “prevention” refer to a method for barring a mammal from acquiring a CTK-related disorder in the first place.

The term “administration” and variants thereof (e.g., “administering” a compound) in reference to a compound means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The above referenced CTK-related disorder may be a cancer selected from, but not limited to, astrocytoma, basal or squamous cell carcinoma, brain cancer, glioblastoma, bladder cancer, breast cancer, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyoma, melanoma, non-small cell lung cancer, neural cancer, ovarian cancer, pancreatic cancer, prostate cancer, renal cell carcinoma, rhabdomyosarcoma, small cell lung cancer, thyoma, thyroid cancer, testicular cancer and osteosarcoma in a further aspect of this invention. More preferably, the CTK-related disorder is a cancer selected from brain cancer, breast cancer, prostate cancer, colorectal cancer, small cell lung cancer, non-small cell lung cancer, renal cell carcinoma or endometrial carcinoma.

As used herein, the term “pharmaceutically acceptable carrier” refers to a carrier medium that does not interfere with the effectiveness of the biological activity of the active ingredient, is chemically inert and is not toxic to the patient to whom it is administered.

### BRIEF DESCRIPTION OF THE DRAWINGS

FIGS. 1a through 1c show the domain structures of Tec kinases and PLCγ1. 1(a) Sequences surrounding Y783 in PLCγ1 and Y180 in Itk SH3. Tyrosine residues that are phosphorylated by Itk are boxed. 1(b) Domain structure of Itk and specific Itk constructs used in this study. The autophosphorylation site of Itk, Y180 within the SH3 domain, is indicated. The domain structures of Tec and Btk are the same as Itk and the Tec and Btk domain fragments used in this study follow the same nomenclature (Btk SH3, Btk 32 (each contain Y223) Tec SH3 and Tec 32 (each contain Y187)). 1(c) The domain structure of PLCγ1 contains a PH domain, EF hand motif, the catalytic domain (comprising the X and Y domains), two SH2 domains (SH2N and SH2C for amino- and carboxy terminal respectively), SH3 domain, a split PH domain indicated by (P and H) and the C2 domain. The PLCγ1 fragments used in this study are shown and substrate fragments each include Y783, the target of Itk kinase activity.

FIGS. 2a through 2d show that Tec mediated phosphorylation requires the SH2 domain adjacent to the target tyrosine. 2(a) Only Itk fragments that contain the SH2 domain and Y180 are phosphorylated by Itk. Ten μM each of Itk SH3, Itk SH3-Linker (Itk 3Linker), Itk SH3-SH2 (Itk 32) or Itk SH3-SH2 with a phenylalanine in place of the Y180 phosphorylation site (Itk 32(Y180F)) were incubated with 250 nM FLAG-tagged Itk full-length (FLItk) enzyme in an in vitro kinase assay. Lanes 5-8, are no enzyme controls. Phosphorylation on Y180 in the SH3 domain is detected with an anti-pY223 Btk antibody (denoted anti-pY180 throughout). Throughout, Coomassie or Ponceau S stain of the gel, anti-FLAG blots and anti Itk blot (bottom panels) show protein levels. For panels a-c, Coommassie stain of the SH3 domain fragments is consistently less efficient than the larger constructs but protein amounts across these experiments are uniform based on measured absorbances. Asterisks next to Commassie stained gel indicate position of Itk 32, Itk 3Linker and Itk SH3. 2(b) Phosphorylation of the Tec and Btk substrates was carried out in a manner similar to that shown in (a) for Itk. Btk SH3 (5 and 10 μM respectively in lane 1-2) or Btk SH3-SH2 (Btk 32) (5 and 10 μM respectively in lanes 3, 4) were incubated with 100 nM FLAG-tagged Btk full-length enzyme in an in vitro kinase assay. Tec SH3 (5 and 10 μM respectively in lanes 5, 6) or Tec SH3-SH2 (Tec 32) (5 and 10 μM respectively in lanes 7, 8) were incubated with 100 nM FLAG-tagged Tec full-length enzyme in an in vitro kinase assay. Lanes 9-12 are the no enzyme controls at substrate concentrations of 10 μM. Phosphorylation on Y223 in the Btk SH3 and Y187 in Tec SH3 domain are detected with an anti-pY223 Btk antibody. Asterisks next to Commassie stained gel indicate position of Tec 32, Btk 32, Btk SH3 and Tec SH3. 2(c) Efficient phosphorylation of PLCγ1 Y783 by Itk occurs only when the adjacent PLCγ1 SH2 domain is present. Lane 1 is enzyme alone control. Varying concentrations (1, 3, 5 and 10 μM in lanes 2-5, respectively) of the fragment of PLCγ1 that contains both the SH2C domain and the following 33 amino acids (PLCγ1 SH2C-Y783-) or the PLCγ1 fragment that contains the 33 residue linker followed by SH3 domain (PLCγ1-Y783-SH3, 1, 3, 5 and 10 μM in lanes 6-9, respectively) were subjected to in vitro phosphorylation by 1.2 μM FLAG-tagged Itk full-length enzyme. Phosphorylation on Y783 in the PLCγ1 constructs is detected using an anti-pY783 PLCγ1 antibody. 2(d) 2 μM purified FLAG-tagged Itk kinase domain alone (Itk KD) was incubated with 1 μM (lane 2) or 10 μM (lane 3) Itk 32 domain in an in vitro kinase reaction as before. Lane 1 is 10 μM Itk 32 domain alone with no enzyme. All data shown are representative of at least three independent experiments.

FIGS. 3a through 3g show that the Itk kinase domain interacts directly with PLCγ1 and Itk SH2 domains in a phosphotyrosine independent manner. 3(a) Empty anti-FLAG resin (lane 1) or purified FLAG-tagged Itk kinase domain (Itk KD) immobilized on an anti-FLAG-resin (lanes 2 and 3) were incubated with purified myc-tagged control protein (lane 2) or myc-tagged Itk SH2 domain (lanes 1 and 3) in a pull-down assay. Blotting with an anti-Myc antibody reveals a direct interaction between purified Itk kinase domain and the purified Itk SH2 domain (lane 3). 3(b) Purified FLAG-tagged Itk KD (lane 1) was incubated with GST (lane 2) or GST-PLCγ1 C-terminal SH2 domain (GST-PLCγ1 SH2C) (lane 3) each immobilized on glutathione beads in a pull-down assay. Anti-FLAG antibody reveals a direct interaction between the Itk kinase domain and the C-terminal SH2 domain of PLCγ1. 3(c) Interaction between the Itk kinase domain and Itk SH2 domain leading to phosphorylation of substrate does not involve the phosphotyrosine binding pocket of the SH2 domain. One μM Itk 32 (lanes 1 and 3) or Itk 32 (R265K) (lanes 2 and 4), were incubated with 250 nM FLAG-tagged full-length Itk in an in vitro kinase assay. Lanes 1 and 2 are no enzyme control. Antibody specific for Itk pY180 shows that the conserved arginine in the phosphotyrosine binding pocket of the SH2 domain is not required. 3(d-e) Effective phosphorylation of PLCγ1 Y783 by the Itk kinase domain does not require the phosphotyrosine binding pocket of the PLCγ1 SH2 domain. One, 3, 5 and 10 μM PLCγ1 SH2C-Y783-(d) or PLCγ1 SH2C(R964A,R696A)-Y783-3(e), were subjected to phosphorylation by 1.2 μM full-length FLAG-tagged Itk. Lanes 5 and 6 (containing 1 and 10 μM substrate, respectively) are no enzyme controls. Coomassie stain of the gels and anti-FLAG blots (bottom panels) show protein levels. (f & g) Interaction between the SH2 domain and Itk kinase domain is independent of the phosphorylation status of the kinase domain. 3(f) FLAG-tagged Itk KD that is expressed alone (lane 1) does not react with a general phosphotyrosine (4G10) antibody while Itk co-expressed with Lck (lane 2) is phosphorylated. Itk KD that is co-expressed with Lck but also pretreated with alkaline phosphatase is not phosphorylated (lane 3). 3(g) Purified FLAG-tagged Itk KD that is either expressed alone (lanes 1 & 2), co-expressed with Lck (lanes 3 & 4) or treated with alkaline phosphatase (lanes 5 & 6) were incubated with immobilized GST (lanes 1, 3 and 5) or immobilized GST-PLCγ1 SH2C, (lanes 2, 4 and 6) in a pull-down assay. An anti-FLAG blot reveals binding of Itk KD to GST-PLCγ1 SH2C and not to GST alone. Ponceau S staining of the membrane (bottom panel) shows the protein levels. All data shown are representative of at least three independent experiments.

FIGS. 4a through 4d demonstrate that the presence of isolated SH2 domain diminishes phosphorylation of Itk Y180 or PLCγ1 Y783. Substrate used in each experiment is indicated above each set of blots. 4(a) (left) Itk 32 substrate was incubated with Itk full-length enzyme as before (lane 1), or with Itk 32 domain:free Itk SH2 domain in ratios of 1:10, 1:25 or 1:50 (lanes 2-4, respectively) or equivalent ratios of PI3K N-terminal SH2 domain (lanes 5-7) and subjected to an in vitro kinase assay. Anti-pY180 antibody shows that free Itk SH2 domain competes with the Itk 32 substrate while the SH2 domain from PI3K does not. Coomassie stain and an anti-FLAG blot (bottom panels) show protein levels. (right) The PLCγ1 SH2 domain competes with the PLCγ1 substrate in a similar manner. Lane 1 contains only Itk enzyme and PLCγ1 SH2C-Y783-substrate without additional free SH2 domain. Ratios of free SH2 domain to substrate are 1:5, 1:10 or 1:50 in lanes 2-4, respectively and equivalent ratios of PI3K SH2 domain are used in lanes 5-7. Coomassie stain of the gel and an anti-Itk blot (bottom panels) show protein levels. 4(b) Same experiment as shown in (a) using the Grb2 SH2 domain as a control. (left) Itk 32 substrate was incubated with Itk full-length enzyme (lane 1), or with Itk 32 domain:free Itk SH2 domain in ratios of 1:10 and 1:25 (lanes 2 & 3) or equivalent ratios of Grb2 SH2 domain (lanes 4 & 5) and subjected to an in vitro kinase assay. (right) Identical experiment following PLCγ1 substrate phosphorylation in the presence of the PLCγ1 and Grb2 SH2 domains. Lane 1 is PLCγ1 substrate alone; lanes 2 & 3 are PLCγ1 substrate:PLCγ1 SH2 domain in a 1:10 and 1:25 ratio, respectively and lanes 4 & 5 are identical ratios of PLCγ1 substrate:Grb2 SH2 domain. 4(c) The Itk SH2 domain competes with autophosphorylation of Y180 within full length Itk. For lanes 2-4, the amount of free SH2 domain is the same as used in panel (a), lanes 2-4. No exogenous SH2 domain has been added in lane 1 and full length Itk is both the enzyme and the substrate. Coomassie stain and an anti-FLAG blot (bottom panels) show protein levels. 4(d) Free Itk SH2 domain does not compete with phosphorylation of a peptide substrate. Initial velocities are shown for phosphorylation of peptide B (sequence shown in FIG. 5a) by full length Itk in the absence (column 1) and presence (column 2 and 3) of Itk SH2 domain. The amount of SH2 domain used here is the same as that used in lanes 3 and 4 in (c).

FIGS. 5a through 5e show that the PLCγ1 C-terminal SH2 domain can mediate docking of Peptide B into the kinase active site of Itk. 5(a) Construction of a PLCγ1 SH2C-Peptide B fusion protein was based on alignment of Y783 within the PLCγ1 linker and the Tyr of Peptide B. The 14 amino acids of Peptide B were used to replace the last 16 amino acids of PLCγ1 SH2C-Y783-. The Tyr that is phosphorylated in each substrate is boxed. A mutant construct is also shown that contains serine in place of the tyrosine of peptide B. This site was mutated to serine instead of phenylalanine due to solubility problems associated with the Tyr to Phe mutation. 5(b) FLAG-tagged Itk LKD enzyme (1 μM) was incubated with 20 μM of the PLCγ1 SH2C-Peptide B fusion protein (lane 4) or 20 μM of the PLCγ1 SH2C-Peptide B(YS) mutant protein (lane 5) in an in vitro kinase assay. Lanes 1-3 are Itk enzyme alone, PLCγ1 SH2C-Peptide B substrate alone and PLCγ1 SH2C-Peptide B(YS) substrate alone, respectively, that have each been subjected to the same in vitro kinase assay conditions. (c & d) FLAG-tagged Itk LKD enzyme was incubated with increasing amounts of biotinylated PLCγ1 SH2C-Peptide B 5(c) or biotinylated Peptide B alone 5(d) in an in vitro kinase assay. 5(e) Kinetic parameters describing phosphorylation of Peptide B and the PLCγ1 SH2C-Peptide B fusion protein by Itk. Data shown are the average of two independent experiments.

FIGS. 6a through 6c show the efficient phosphorylation of Itk Y511 by Lck requires the Itk SH2 domain. 6(A) Schematic of the Itk constructs used as substrates in this study. 6(B) Differential in vivo phosphorylation of Itk fragments by Lck. Catalytically inactive (K390R) forms of Itk SH2-kinase domain (2KD) or Itk kinase domain alone (KD), respectively were expressed without (lanes 1 and 2) or with (lanes 3 and 4) full-length wild-type Lck in Sƒ9 cells. The cell lysates were normalized for protein expression and western blotted with a Btk phospho Y551 (to detect Itk phospho Y511), anti-FLAG and anti-Lck antibodies. 6(C) Differential in vitro phosphorylation of Itk fragments by Lck. Purified, catalytically inactive forms of Itk SH2-kinase domain (2KD) (lanes 1, 4 and 7), Itk linker kinase domain (LKD) (lanes 2, 5 and 8) or Itk kinase domain (KD) (lanes 3, 6 and 9) were incubated with immunoprecipitated full-length Lck (lanes 4, 5 and 6) or Lck kinase domain alone that was purified from bacteria (lanes 7, 8 and 9) in a kinase assay buffer. Lanes (1, 2 and 3) are no enzyme controls. The samples were boiled and western blotted with the respective antibodies as before. Lanes 7,8 & 9 do not show the Lck kinase domain because the antibody is to the truncated N-terminus.

FIG. 7 demonstrates the direct interaction between the Lck kinase domain and the Itk SH2 domain. Purified His-tagged wild type (lanes 1, 3 and 5) or kinase inactive (lanes 2, 4 and 6) Lck kinase domain was incubated with Myc and His-tagged Itk SH2 R265K mutant (lanes 3 and 4) or wild type Itk SH2 domain (lanes 5 and 6). The Itk SH2 domains were immunoprecipitated using an anti-Myc antibody and the samples were western blotted with an anti-His antibody to detect the presence of the Lck kinase domain. Lanes 1 and 2 are beads alone control.

FIGS. 8a and 8b show that the Itk SH2 domain can mediate docking of Lck Peptide substrate into the kinase active site of Lck. (8A) Schematic showing the Lck peptide substrate fusions used in the study. The Lck peptide substrate (YIYGSFK) was fused to the Itk SH2 domain at either the N-terminus or the C-terminus with poly Glycine linkers of variable (5, 6 or 10 amino acids) length. The phosphorylated tyrosine is shown in bold. (8B) 10 μM of the SH2 Lck peptide fusions or 400 μM of the peptide substrate alone were incubated with Lck kinase domain in an in vitro kinase assay. Activity was measured by capturing the substrates onto a P81 or streptavidin membrane and counted by scintillation counting. Lane 1 is enzyme alone, lane 2: Itk SH2 domain alone, lane 3: N5, lane 4: N10, lane 5: C5, lane 6: C5r, lane 7: C6, lane 8: C6r, lane 9: peptide substrate (YIYGSFK), and lane 10: Biotinylated peptide substrate YIYGSFK. Data shown are the average of two independent experiments. Note that peptide concentrations in lanes 9 and 10 are 400 μM while protein concentrations in other lanes are only 10 μM.

FIG. 9 shows molecular determinants of the Itk SH2 mediated docking interaction. (9A) SH3-SH2 substrate is used as shown previously (see FIGS. 2a, 4a & b). The substrate variants are MycHis-tagged. One or five μM SH3-SH2 substrate (wild type, lanes 3, 4) or indicated mutants (lanes 5-20) were subjected to in vitro phosphorylation by 250 nM full length FLAG-tagged Itk enzyme. Lanes 1 & 2 are no Itk enzyme controls. Anti-pY180 blot indicates extent of SH3-SH2 substrate phosphorylation by Itk, anti-Myc blot shows SH3-SH2 substrate levels and anti-FLAG blot shows the Itk enzyme levels. Efficient phosphorylation of Y180 within the SH3-SH2 substrate by Itk occurs only when the Itk kinase domain:Itk SH2 domain docking interaction is intact. Mutations that disfavor or disrupt the docking interaction lead to diminished Y180 phosphorylation levels. (Asterisks next to top panel indicate an impurity.) (9B) Primary amino acid sequence of the Itk SH2 domain showing the eleven residues that upon mutation to alanine disrupt the substrate docking mechanism. Sequence numbering is indicated and is derived from the full-length Itk mouse sequence. (9C) Structure of Itk SH2 domain (pdb: 1LUN) showing the location of the eleven residues found to mediate substrate docking with the Itk kinase domain in red stick representation. (9D) (top) Surface representation of the Itk SH2 structure showing the contiguous nature of the docking interaction site. The surface defined by the eleven residues mutated in panel A is indicated in red and is located on the face of the SH2 domain that is opposite the well characterized phosphotyrosine binding pocket of the SH2 domain (indicated by dashed circle on the backside of the domain (lower part of FIG. 9D)). This is consistent with our earlier observation (see FIG. 3c and e) that mutation of the phosphotyrosine binding pocket has no effect on the substrate docking interaction. It is expected that the homologous sites in the SH2 domains of the other Tec kinases (Btk, Tec, Bmx and Rlk) will mediate substrate docking to the respective kinase domains.

FIG. 10 shows specific amino acids of the PLCγ1 SH2 domain that mediate substrate phosphorylation. (10A) Indicated mutations were introduced into the PLCγ1 SH2C-Y783-substrate used previously (see Example 1; also indicated here as PLCγ1 SH2 linker substrate). One or five μM PLCγ1 SH2 linker substrate (wild type, lanes 2, 3, 4) or indicated mutants (lanes 5-22) were subjected to in vitro phosphorylation by 700 nM full length FLAG-tagged Itk enzyme. Lane 1 is enzyme alone control. Lanes 4, 7, 10, 13, 16, 19 and 22 are no Itk enzyme controls for the wild type substrate or indicated mutants. Anti-pY783 blot indicates extent of PLCγ1 SH2 linker substrate phosphorylation by Itk; Coomassie Stain shows substrate levels and anti-FLAG blot shows the Itk enzyme levels. Efficient phosphorylation of Y783 within the substrate by Itk occurs only when the Itk kinase domain:Itk SH2 domain docking interaction is intact. Mutation of SH2 residues K695, R696, S699, K749 or R753 to alanine disfavors or disrupts the docking interaction leading to diminished Y783 phosphorylation levels. (10B) Primary amino acid sequence of the PLCγ1 SH2 showing the five residues that upon mutation to alanine disrupt the substrate docking mechanism. Sequence numbering is indicated and is derived from the full-length PLCγ1 rat sequence. (10C) Structure of PLCγ1 SH2 domain (pdb: 2pld) showing the location of the five residues found to date to mediate substrate docking with the Itk kinase domain in red stick representation. (10D) Surface representation of the PLCγ1 c-terminal SH2 structure showing the surface of the docking interaction site. The surface defined by the five residues mutated in panel A is indicated in red and is located on the face of the SH2 domain. Methods are as described for Example 1.

FIG. 11 is a diagram showing the screening of small molecule libraries: Adapted from http://iccb.med.harvard.edu/

FIG. 12 is a schematic of SH2 mediated phosphorylation events within the Tec and Src CTK families. These intermolecular kinase-substrate complexes are excellent and unique targets for small molecules (or other entities) that could interfere with complex formation and substrate phosphorylation. Given the novel nature of the SH2 mediated substrate docking interaction, this approach provides an alternative to inhibitors directed toward the active site that will exhibit significant cross reactivity with other protein kinases.

### DETAILED DESCRIPTION OF THE PREFERRED EMBODIMENT

The present invention relates to drug screening and candidate selection as well as therapeutic protocols and pharmaceutical compositions designed to modulate non-receptor mediated tyrosine protein kinase cascades. The invention further relates to methods of modulating immune activity, cellular proliferation and the like by modulating the non receptor tyrosine protein kinase interaction via means of a novel SH2 substrate docking system that has been found to be required for tyrosine phosphorylation.

The invention is based, in part, on the Applicants' discovery that remote SH2 domains in substrates of CTKs are required to achieve tyrosine phosphorylation. Applicants have identified a previously unrecognized docking role for the SH2 domain within the substrates of the CTK's that facilitate phosphorylation at a remote tyrosine. In some instances, a tyrosine kinase creates a phosphotyrosine containing binding site on the substrate for the kinase's own internal SH2 domain. The ensuing kinase derived SH2-substrate interaction leads to phosphorylation at additional sites on the substrate. In contrast to the well-characterized role of the SH2 domain in this type of processive phosphorylation mechanism, Applicant's have identified that SH2 domains within some substrates of the tyrosine kinases harbor a recognition motif that is required for efficient and selective phosphorylation of the target tyrosine.

Src Homology 2 (SH2) domains are well known motifs found in many proteins involved in signal transduction. SH2 domains are modules of about 100 amino acids in size that are known for their ability to specifically recognize the phosphorylated state of tyrosine residues, thereby allowing SH2 domain-containing proteins to localize to tyrosine-phosphorylated sites. For a review of SH2 motifs, sequences and descriptions, see, Pawson et al, Cell, volume 16 pages 191-203, June 1994, the disclosure of which is incorporated herein by reference. SH2 domains of various proteins are also listed in the website www.proteoscape.uchicago.edu/sh2/main_table.html. Such domains are easily identifiable by these and other resources (sequence alignment) available to those of skill in the art.

Quite in contrast to the well-characterized canonical phosphotyrosine ligand recognition described above, the substrate docking interaction described by the Applicants is mediated by interactions that do not involve a phosphotyrosine residue. Neither the kinase domains of the enzymes nor the SH2 domains of the substrates need to be phosphorylated on tyrosine or any other amino acid. Specific examples of the SH2/kinase docking interaction are as follows. The direct substrate of Itk (a Tec family CTK) in T cells, PLCγ1, harbors an SH2 domain (SH2C) that binds directly to the Itk kinase domain allowing the PLCγ1 tyrosine (Tyr 783) to be efficiently phosphorylated by Itk. In a similar manner the direct substrate of the Src family kinase Lck is Tyr 511 in Itk. The Itk Tyr 511 substrate requires the presence of the remote SH2 domain in Itk for efficient phosphorylation by the Lck kinase, As well, autophosphorylation of Tyr 180 in the Itk SH3 domain depends on Itk SH2 mediated interactions with the Itk kinase domain. This mode of substrate recognition is evident within the full-length enzyme as addition of competing exogenous Itk SH2 domain to full length Itk completely eliminates Itk autophosphorylation. This direct kinase domain—SH2 interaction is a novel mode of substrate recognition that provides for alternative strategies for inhibiting the CTK signal cascade for modulating T-cell mediated immunity and other cellular proliferative and regulatory functions achieved by the CTKs. Unlike canonical SH2/phosphotyrosine binding, the SH2 domains in these examples of substrate docking mediate their effect in a phosphotyrosine-independent manner.

In one embodiment the invention includes methods for modulating the CTK signal cascade by providing additional SH2 domains for the CTK SH2 docking interaction. For example, applicants have shown that the addition of exogenous SH2 domains to a kinase reaction results in direct competition with the substrate SH2 domain and thus decreases CKT phosphorylation. One embodiment thus includes methods and pharmaceutical compositions which include contacting cells with agents containing SH2 motifs for competitive inhibition of the CTK pathway, and thereby decreasing the CTK mediated responses in the cell for treatment of CTK related disorders or conditions.

Yet another embodiment provides methods and compositions for increasing the substrate CTK interaction by engineering substrates to contain additional SH2 domains to encourage the CTK SH2 docking and subsequent phosphorylation of the substrate. Applicants demonstrated that the kinetic parameters of phosphorylation of CTK substrates are improved by fusing the peptide substrates to SH2 domains. The invention includes methods for increasing the CTK cascade for boosting immune responses and the like by including pharmaceutical compositions and methods using a modified CTK substrate engineered with one or more additional SH2 domains.

In a preferred embodiment the invention comprises cell-based and animal model based screening assays to identify novel agents that modulate CTK and its interaction and/or activation of cellular components of the non-receptor mediated protein kinase signaling cascade. In addition, the present invention relates to screening assays to identify novel agents that inhibit substrate SH2 mediated activation of the same. A variety of protocols and techniques may be utilized to screen for agents that modulate, or preferably interfere with and/or inhibit the interaction of the novel substrate SH2 domain kinase protein interaction disclosed herein.

### Screening for Modulators of the CTK Docking Mechanism

The present invention further comprises methods for identifying modulators, preferably inhibitors of CTK signaling by interfering with the novel docking mechanism of the invention activity. While the discussion herein is made in the context of inhibitors, it shall be understood that certain embodiments may involve the up-regulation of such mechanism and the same strategies can be employed to determine such modulators as well.

CTKs and their substrates may be used as a target in screening for compounds that inhibit, decrease or down-regulate kinase activity in cells via the docking mechanism described herein. These assays may comprise random screening of large libraries of candidate substances. Alternatively, the assays may be used to focus on particular classes of compounds selected with an eye towards structural attributes that are believed to make them more likely to inhibit the protein-protein interaction of CTKs and their SH2 docking substrates necessary for function. By function, it is meant that one may assay for inhibition of the ability of the CTK enzyme to attach phosphate groups onto the substrate. Alternatively, one may assay for ability of a substance to alter the protein-protein interaction function that mediates substrate docking.

To identify a CTK inhibitor, one generally will determine CTK activity or downstream cellular effects of CTK activity (e.g. cytokine production) in the presence or absence of the candidate substance, wherein an inhibitor is defined as any substance that down-regulates, reduces, inhibits or decreases CTK activity due to its effect on the CTK substrate docking mechanism. For example, a method may generally comprise: a) providing a cell; b) contacting the cell with a candidate compound; and c) assessing the effect of the candidate compound on CTK docking activity, wherein a decrease in the amount of CTK docking activity, as compared to the amount of CTK activity in a similar cell not treated with the candidate compound, indicates that the candidate compound has anti-cancer activity or inhibition of immune cell mediated immunity. An alternative approach to identify a CTK inhibitor will be to monitor the effect of candidate substances on the protein-protein interaction required for docking and substrate phosphorylation. In this instance the assay will be a binding assay and not an enzymatic assay.

Assays may be conducted in cell free systems, in isolated cells, or in organisms including transgenic animals. It will, of course, be understood that all the screening methods of the present invention are useful in themselves notwithstanding the fact that effective candidates may not be found.

### Inhibitors

As used herein the term “candidate substance” or “candidate compound” refers to any molecule that may potentially modulate the activity of the CTK/substrate docking mechanism herein described. A CTK inhibitor, may be a compound that overall affects an increase or decrease in CTK docking activity away from the kinase active site, which may be accomplished by any compound or molecule described in the methods and compositions herein may be a modulator of CTK docking activity.

The candidate substance may be a protein or fragment thereof, a small molecule, carbohydrate or nucleic acid molecule. It may prove to be the case that the most useful pharmacological compounds will be compounds that are structurally related to CTK, its substrates, other protein tyrosine kinases, or that binds CTK. Using lead compounds to help develop improved compounds is known as “rational drug design” and includes not only comparisons with known inhibitors, but predictions relating to the structure of target molecules.

Candidate compounds or inhibitors of the present invention will likely function to inhibit, regions necessary for activity of CTK docking in a cancer,immune or normal cell. Such candidate compounds may be inhibitors or regulators of protein tyrosine kinases; or may likely be involved in controlling cellular proliferation in a cancer or tumor cell. These candidate compounds may be, a protein or fragment thereof, nucleic acids, antibodies (including single chain antibodies), or organopharmaceuticals, but are not limited to such.

### Rational Drug Design

The present invention also provides methods for developing drugs that inhibit CTK activity by inhibiting the docking mechanism necessary for activity. Such drugs may be used to treat a cancer, an immune disorder or any other CTK disease,or suppress a normal immune response. One such method involves the prediction of the three dimensional structure of a validated protein tyrosine kinase docking target, or the docking domain itself, using molecular modeling and computer stimulations of the docking areas. The resulting structure may then be used in docking studies to identify potential small molecule inhibitors that bind, not in the enzyme's active site but in the essential SH2 substrate docking site, or the SH2 docking module itself, with favorable binding energies. Inhibitors identified may then be tested in biochemical assays to further identify CTK drug targets for CTK disease treatment.

Rational drug design is therefore used to produce structural analogs of substrates for CTK. By creating such analogs, it is possible to fashion drugs that are more active or stable than the natural molecules, which have different susceptibility to alteration or which may affect the function of various other molecules. In one approach, one would generate a three-dimensional structure for the CTK docking targets of the invention or a fragment thereof. This could be accomplished by X-ray crystallography, NMR spectroscopy, computer modeling or by a combination of approaches.

It also is possible to use antibodies to ascertain the structure of a docking target compound inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods known in the art and described herein for producing antibodies, using an antibody as the antigen.

On the other hand, one may simply acquire, from various commercial sources, small molecule libraries that are believed to meet the basic criteria for useful drugs in an effort to identify useful compounds. Screening of such libraries, including combinatorially generated libraries (e.g., peptide libraries), is a rapid and efficient way to screen large numbers of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, carbohydrate, polynucleotide, small molecule inhibitors or any other compounds that may be designed through rational drug design starting from known inhibitors or stimulators.

In addition to the inhibiting compounds initially identified, the inventors also contemplate that other sterically similar compounds may be formulated to mimic the key portions of the structure of the inhibitors. Such compounds, which may include peptidomimetics of peptide inhibitors, may be used in the same manner as the initial inhibitors.

The term “drug” is intended to refer to a chemical entity, whether in the solid, liquid, or gaseous phase which is capable of providing a desired therapeutic effect when administered to a subject. The term “drug” should be read to include synthetic compounds, natural products and macromolecular entities such as polypeptides, polynucleotides, carbohydrates or lipids and also small entities such as neurotransmitters, ligands, hormones or elemental compounds. The term “drug” is meant to refer to that compound whether it is in a crude mixture or purified and isolated.

### Bioisosterism

The present invention also contemplates the application of bioisosterism, the concept of isosterism to modify biological activity of a lead compound, in developing drugs that can inhibit CTK or substrate docking activity that may be used as therapeutic agents. As discussed above, a lead compound with a desired pharmacological activity may have associated with it undesirable side effects, characteristics that limit its bioavailability, or structural features which adversely influence its metabolism and excretion from the body. Bioisosterism represents one approach used in the art for the rational modification of lead compounds into safer and more clinically effective agents (Patani and LaVoie, 1996). The ability of a group of bioisosteres to elicit similar biological activity has been attributed to common physicochemical properties such as electro-negativity, steric size, and lipophilicity. Bioisosteric replacements of functional groups based on the understanding of the pharmacophore and the physicochemical properties of the bioisosteres have enhanced the potential for the successful development of new clinical agents. A critical component for bioisosterism is that bioisosteres affect the same pharmacological target as agonists or antagonists and, thereby, have biological properties that are related to each other.

Bioisosteres are classified as either classical or nonclassical. Classical bioisosteres have been traditionally divided into several distinct categories: (a) monovalent atoms or groups; (b) divalent atoms or groups; (c) trivalent atoms or groups; (d) tetrasubstituted atoms; and (e) ring equivalents. Nonclassical bioisosteres can be divided into (a) rings vs noncyclic structures; and (b) exchangeable groups. Nonclassical isosteres differ from that of the classical bioisosteres in that they do not obey the steric and electronic definition of classical isosteres. A notable characteristic of nonclassical bioisosteres is that they do not have the same number of atoms as the substituent or moiety for which they are used as a replacement.

### Tyrosine Kinase Assays

Assays that measure the phosphorylation of proteins or peptides containing tyrosine may also be employed in the present invention. These are quite common and are commercially available from a number for sources. The most common method involves measuring the transfer of ³²P to tyrosine. Such assays are commercially available from suppliers such as Promega (Madison, Wis.) or Applied Biosystems (Foster City, Calif.). Hoffman La Roche offers a non radioactive assay kit which utilizes a specific peptide substrate that is biotinylated at the amino terminus. The enzyme reaction is quenched by the kinase inhibitor piceatannol. The substrate is then immobilized on streptavidin coated microtiter plates and the reaction mixture is washed out. The fraction of phosphorylated substrate is detected by scintillation counting (or immunochemically by a highly specific antibody conjugated to peroxidase). The rate of substrate phosphorylation can be readily quantified. Other assays are also available from Cal Biochem (San Diego).

### In Vitro Assays

A quick, inexpensive and easy assay to run is an in vitro assay. Such assays generally use isolated molecules, and can be run quickly and in large numbers, thereby increasing the amount of information obtainable in a short period of time. A variety of vessels may be used to run the assays, including test tubes, plates, dishes and other surfaces such as dipsticks or beads.

One example of a cell-free assay is a binding assay. While not directly addressing function, the ability of a compound to bind to a target molecule such as CTK or the SH2 docking module in a specific fashion is strong evidence of a related biological effect, which can be assessed in follow up screens. For example, binding of a molecule to CTK may, in and of itself, be inhibitory, due to steric, allosteric or charge-charge interactions that prevent substrate SH2 docking. The CTK itself, the substrate derived SH2 domain, or the CTK/SH2 substrate complex may be either free in solution, fixed to a support, expressed in or on the surface of a cell. Either the CTK, the substrate derived SH2, or the compound may be labeled, thereby permitting measuring of the binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### In Cyto Assays

The present invention also contemplates the screening of compounds for their ability to inhibit CTK docking activity in cells. Various cell lines can be utilized for such screening assays, including cells specifically engineered for this purpose. Depending on the assay, culture may be required. The cell is examined using any of a number of different physiologic assays that are well known to those of skill in the art.

### In Vivo Assays

In vivo assays involve the use of various animal models, including transgenic animals that have been engineered to have specific defects such as CTK overexpression, or that carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies an inhibitor. The characteristics may be any of those discussed above with regard to CTK function, or it may be broader in the sense of “treating” the condition present in the animal.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve measuring toxicity and dose response can be performed in animals in a more meaningful fashion than in in vitro or in cyto assays.

### Disease Treatment

The present invention embodies a method of treating CTK associated conditions or disease states such as cancer, autoimmune disorders and the like, by the delivery of a CTK modulator, preferably an inhibitor to a subject having need of such treatment. Likewise the invention embodies a method of suppressing an otherwise normal or healthy immune response. Examples of diseases or condition include but are not limited to organ transplantation, cancers, psoriasis, hepatic cirrhosis, diabetes, atherosclerosis, angiogenesis, restenosis, ocular diseases, rheumatoid arthritis and other inflammatory disorders, autoimmune diseases and a variety of renal disorders or any other condition which may be treated by modulating or inhibiting or decreasing the activity of CTK activity such as in organ transplant procedures to reduce immunoreactivity.

### Pharmaceutical Compositions and Routes of Administration

Pharmaceutical compositions of the present invention comprise administering an effective amount of one or more modulators, preferably inhibitors that inhibit or down-regulate the CTK docking activity (and/or an additional agent) dissolved or dispersed in a pharmaceutically acceptable carrier to a subject. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one CTK inhibitor or additional active ingredient will be known to those of skill in the art in light of the present disclosure, and as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

A pharmaceutical composition of the present invention may comprise different types of pharmaceutically acceptable carriers depending on whether it is to be administered in solid, liquid or aerosol form, and whether it needs to be sterile for such routes of administration as injection. A pharmaceutical composition of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kglbody weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kgibody weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

A CTK inhibitor(s) of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain aspects of the invention, the CTK inhibitors are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Combination Therapies with CTK Inhibitor(s)

In order to increase the effectiveness of a cancer treatment with the compositions of the present invention, such as a CTK inhibitor, it may be desirable to combine these compositions with other cancer therapy agents. For example, the treatment of a cancer may be implemented with therapeutic agents of the present invention in conjunction with other anti-cancer therapies. Thus, in the present invention, it is contemplated that a CTK inhibitor(s) may be used in conjunction with a chemotherapeutic, a radiotherapeutic, an immunotherapeutic or other biological intervention, in addition to pro-apoptotic or cell cycle regulating agents or protein tyrosine phosphatase regulators.

This process may involve contacting the cell(s) with a CTK inhibitor and a therapeutic agent at the same time or within a period of time wherein separate administration of the inhibitor and an agent to a cell, tissue or organism produces a desired therapeutic benefit. The terms “contacted” and “exposed,” when applied to a cell, tissue or organism, are used herein to describe the process by which a CTK inhibitor and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both a CTK inhibitor and one or more agents, or by contacting the cell with two or more distinct compositions or formulations, wherein one composition includes a CTK inhibitor and the other includes one or more agents.

The present invention further encompasses pharmaceutical compositions containing the CTK modulating compounds described herein. The therapeutic modalities of the invention further encompass combination therapies in which an agent which interferes with the interaction and/or activation of the substrate SH2 docking mechanism and CTK and at least one other therapeutic agent are administered either concurrently, e.g., as an admixture, separately but simultaneously or concurrently; or sequentially, including cycling therapy. Cycling therapy involves the administration of a first compound (for example an anti-cancer agent) for a period of time and repeating this sequential administration, i.e., the cycle, in order to reduce the development of resistance to one of the therapies.

The novel pharmaceutical combinations of the present invention provide a means of treatment which may not only reduce the effective dose of either drug required for therapeutic activity, thereby reducing toxicity, but may also improve the absolute effect as a result of attacking the cancer through multiple mechanisms.

The specific kinase/SH2 substrate pairs already established in this invention that are therefore immediate targets for modulation are as follows:

| | SUBSTRATES (Tyr that is phosphorylated via an SH2 |
|---|---|
| | dependent docking mechanism is indicated |
| CTK ENZYME | where known.) |
| Lck | Itk (Y511) |
| Lyn | Btk (Y551); Tec (Y519) |
| Fyn | Rlk (Y420) |
| Src | Bmx (Y566) |
| Itk | Itk (Y180); PLCγ1 (Y783); PLCγ2 |
| Btk | Btk (Y223); PLCγ2 (Y759) |
| Tec | Tec (Y188); PLCγ1 (Y783); PLCγ2 (Y753, 759) |
| Bmx | Bmx (Y194); Stat3 (Y705); PLCγ2 |
| Rlk | Rlk (Y91); PLCγ1 (Y783); PLCγ2 |

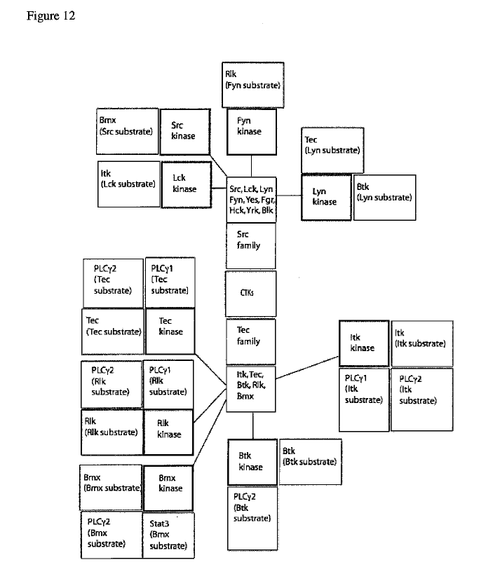

A further list of CTK enzymes and potential SH2 domain substrates that may be used in the invention follows. These lists embody possible kinase/substrate pairs for which SH2 mediated substrate docking is present. In addition, the present invention comprises screening methods to identify specific kinase/SH2 pairs and SH2 mediated docking in functional assays. For any combination of CTK enzyme and SH2 domain (see lists below) it is envisioned that binding assays could be carried out either at the level of individual proteins and/or in a high throughput manner. The identification of a specific CTK/SH2 complex in such screens would be indicative of a SH2 mediated docking mechanism for that kinase enzyme and its substrate(s). The functional role of such SH2 binding to the CTK would be assessed by tagging the SH2 domain with a generic peptide substrate and comparing kinetic parameters (or overall phosphorylation levels by western blot) for the generic peptide alone and that which is covalently attached to the docking SH2 domain. The Applicants demonstrated enhanced tyrosine phosphorylation for two generic peptide substrates (for the Itk and Lck kinases) when the peptide substrate was fused to the Itk SH2 domain. A diagram showing various CTK enzymes and their substrates is shown at FIG. 12.
A list of currently known non-receptor tyrosine kinases (including those already listed above) is as follows:
Adapted from www.expasy.org/cgi-bin/nicezyme.pI?2.7.10.2
Each entry includes: UniProtKB/Swiss-Prot entry, name of kinase₁₃ species;

| | | |
|---|---|---|
| P03949, ABL1_CAEEL; | P00519, ABL1_HUMAN; | P00520, ABL1_MOUSE; |
| P42684, ABL2_HUMAN; | Q4JIM5, ABL2_MOUSE; | P11681, ABL_CALVI; |
| P00522, ABL_DROME; | P10447, ABL_FSVHY; | P00521, ABL_MLVAB; |
| Q07912, ACK1_HUMAN; | O54967, ACK1_MOUSE; | O02742, ACK2_BOVIN; |
| Q46631, AMSA_ERWAM; | P51451, BLK_HUMAN; | P16277, BLK_MOUSE; |
| P51813, BMX_HUMAN; | P97504, BMX_MOUSE; | P08630, BTKL_DROME; |
| Q8JH64, BTK_CHICK; | Q06187, BTK_HUMAN; | P35991, BTK_MOUSE; |
| P39851, CAPB_STAAU; | Q9AHD2, CPSD1_STRPN; | Q54520, CPSD2_STRPN; |
| Q3K0T0, CPSD_STRA1; | Q04663, CPSD_STRA3; | Q9AFI1, CPSD_STRA5; |
| P0C0T9, CPSD_STRAG; | Q0VBZ0, CSK_BOVIN; | P41239, CSK_CHICK; |
| P41240, CSK_HUMAN; | P41241, CSK_MOUSE; | P32577, CSK_RAT; |
| P58593, EPSB1_RALSO; | Q45409, EPSB2_RALSO; | P58764, ETK_ECO27; |
| Q8XC28, ETK_ECO57; | P38134, ETK_ECOLI; | Q00944, FAK1_CHICK; |
| Q05397, FAK1_HUMAN; | P34152, FAK1_MOUSE; | O35346, FAK1_RAT; |
| Q91738, FAK1_XENLA; | Q14289, FAK2_HUMAN; | Q9QVP9, FAK2_MOUSE; |
| P70600, FAK2_RAT; | Q9TTY2, FER_CANFA; | P16591, FER_HUMAN; |
| P70451, FER_MOUSE; | P09760, FER_RAT; | P14238, FES_FELCA; |
| P00542, FES_FSVGA; | P00543, FES_FSVST; | P07332, FES_HUMAN; |
| P16879, FES_MOUSE; | P00544, FGR_FSVGR; | P09769, FGR_HUMAN; |
| P14234, FGR_MOUSE; | P00541, FPS_AVISP; | P18106, FPS_DROME; |
| P00530, FPS_FUJSV; | P42685, FRK_HUMAN; | Q922K9, FRK_MOUSE; |
| Q62662, FRK_RAT; | Q05876, FYN_CHICK; | P06241, FYN_HUMAN; |
| P39688, FYN_MOUSE; | P13406, FYN_XENLA; | P27446, FYN_XIPHE; |
| P08631, HCK_HUMAN; | Q95M30, HCK_MACFA; | P08103, HCK_MOUSE; |
| P50545, HCK_RAT; | P53356, HTK16_HYDAT; | Q08881, ITK_HUMAN; |
| Q03526, ITK_MOUSE; | O12990, JAK1_BRARE; | Q09178, JAK1_CYPCA; |
| P23458, JAK1_HUMAN; | P52332, JAK1_MOUSE; | O60674, JAK2_HUMAN; |
| Q62120, JAK2_MOUSE; | Q62689, JAK2_RAT; | P52333, JAK3_HUMAN; |
| Q62137, JAK3_MOUSE; | Q63272, JAK3_RAT; | Q24592, JAK_DROME; |
| Q10925, KIN25_CAEEL; | P34265, KIN31_CAEEL; | P43405, KSYK_HUMAN; |
| P48025, KSYK_MOUSE; | Q00655, KSYK_PIG; | Q64725, KSYK_RAT; |
| P18160, KYK1_DICDI; | P18161, KYK2_DICDI; | Q5PXS1, LCK_AOTNA; |
| P42683, LCK_CHICK; | P06239, LCK_HUMAN; | P06240, LCK_MOUSE; |
| Q01621, LCK_RAT; | Q95KR7, LCK_SAISC; | P07948, LYN_HUMAN; |
| P25911, LYN_MOUSE; | Q07014, LYN_RAT; | P42679, MATK_HUMAN; |
| P41242, MATK_MOUSE; | P41243, MATK_RAT; | Q9I7F7, PR2_DROME; |
| Q13882, PTK6_HUMAN; | Q64434, PTK6_MOUSE; | O52788, PTK_ACIJO; |
| P23049, SEA_AVIET; | Q86YV5, SG223_HUMAN; | Q571I4, SG223_MOUSE; |
| Q9H792, SG269_HUMAN; | Q69Z38, SG269_MOUSE; | Q24145, SHARK_DROME; |
| P42687, SPK1_DUGTI; | P13115, SRC1_XENLA; | P13116, SRC2_XENLA; |
| Q9V913, SRC42_DROME; | P00528, SRC64_DROME; | P15054, SRC_AVIS2; |
| P00525, SRC_AVISR; | P14084, SRC_AVISS; | P14085, SRC_AVIST; |
| P00523, SRC_CHICK; | P12931, SRC_HUMAN; | P05480, SRC_MOUSE; |
| Q9WUD9, SRC_RAT; | P25020, SRC_RSVH1; | P00526, SRC_RSVP; |
| P31693, SRC_RSVPA; | P00524, SRC_RSVSA; | P63185, SRC_RSVSE; |
| P42686, SRK1_SPOLA; | P42688, SRK2_SPOLA; | P42689, SRK3_SPOLA; |
| P42690, SRK4_SPOLA; | Q9H3Y6, SRMS_HUMAN; | Q62270, SRMS_MOUSE; |
| P17713, STK_HYDAT; | Q6J9G0, STYK1_HUMAN; | Q6J9G1, STYK1_MOUSE; |
| P42680, TEC_HUMAN; | P24604, TEC_MOUSE; | Q13470, TNK1_HUMAN; |
| Q99ML2, TNK1_MOUSE; | P42681, TXK_HUMAN; | P42682, TXK_MOUSE; |
| P29597, TYK2_HUMAN; | Q9R117, TYK2_MOUSE; | P47817, WEE1A_XENLA; |
| P0C158, WEE1B_HUMAN; | Q66JT0, WEE1B_MOUSE; | P54350, WEE1_DROME; |
| P30291, WEE1_HUMAN; | P47810, WEE1_MOUSE; | Q63802, WEE1_RAT; |
| Q8X7L9, WZC_ECO57; | P76387, WZC_ECOLI; | Q8Z5G6, WZC_SALTI; |
| Q9F781, WZC_SALTY; | Q48452, YC06_KLEPN; | P00527, YES_AVISY; |
| Q28923, YES_CANFA; | P09324, YES_CHICK; | P07947, YES_HUMAN; |
| Q04736, YES_MOUSE; | P10936, YES_XENLA; | P27447, YES_XIPHE; |
| Q02977, YRK_CHICK; | P71051, YVEL_BACSU; | P96716, YWQD_BACSU; |
| Q11112, YX05_CAEEL; | P43403, ZAP70_HUMAN; | P43404, ZAP70_MOUSE; |

A list of Known human and mouse SH2 domains (including those found in substrates already listed above (shown in bold)) is as follows:

Table modified From www.proteoscape.uchicago.edu/sh2/index.html a project of the Nash lab at the University of Chicago.

| | Gene | | | Genomic |
|---|---|---|---|---|
| Name | ID | Domains | Alternate Names | Location |
| ABL1 | 25 | 1 | ABL, JTK7, | 9q34.1 |
| | | | p150, c-ABL | |
| ABL2 | 27 | 1 | ARG, ABLL | 1q24–q25 |
| APS | 10603 | 1 | | 7q22 |
| BCAR3 | 8412 | 1 | SH2D3B, NSP2, | 1p22.1 |
| | | | KIAA0554 | |
| BKS | 55620 | 1 | STAP2, | 19p13.3 |
| | | | FLJ20234 | |
| BLK | 640 | 1 | MGC10442 | 8q23–p22 |
| BLNK | 29760 | 1 | Ly57, SLP65, | 10q23.2–q23.33 |
| | | | BLNK-s, SLP-65 | |
| BMX | 660 | 1 | ETK, PSCTK2, | Xp22.2 |
| | | | PSCTK3 | |
| BRDG1 | 26228 | 1 | STAP-1 | 4q13.2 |
| BRK | 5753 | 1 | PTK6 | 20q13.3 |
| BTK | 695 | 1 | AT, ATK, BPK, | Xq21.33–q22 |
| | | | XLA, IMD1, | |
| | | | AGMX1, | |
| | | | PSCTK1 | |
| CBL | 867 | 1 | CBL2 | 11q23.3 |
| CBLB | 868 | 1 | RNF56 | 3q13.11 |
| CBLC | 23624 | 1 | CBL-3, CBL-SL, | 19q13.2 |
| | | | RNF57 | |
| CHN1 | 1123 | 1 | CHN, | 2q31–q32.1 |
| | | | ARHGAP2, | |
| | | | RHOGAP2 | |
| CHN2 | 1124 | 1 | ARHGAP3, | 7p15.3 |
| | | | RHOGAP3 | |
| CISH | 1154 | 1 | CIS, G18, SOCS, | 3p21.3 |
| | | | CIS-1 | |
| CRK | 1398 | 1 | CRKII | 17p13.3 |
| CRKL | 1399 | 1 | Crk-like | 22q11.21 |
| CSK | 1445 | 1 | CYL | 15q23–q25 |
| DAPP1 | 27071 | 1 | BAM32, | 4q25–q27 |
| | | | DKFZp667E0716 | |
| FER | 2241 | 1 | TYK3 | 5q21 |
| FES | 2242 | 1 | FPS, c-FER, p94- | 15q26.1 |
| | | | FER | |
| FGR | 2268 | 1 | Src2, c-fgr, p55c- | 1p36.2–p36.1 |
| | | | fgr | |
| FRK | 2444 | 1 | GTK, RAK, | 6q21–q22.3 |
| | | | PTK5 | |
| FYN | 2534 | 1 | SLK, SYN, | 6q21 |
| | | | MGC45350 | |
| GADS | 9402 | 1 | P38, GADS, | 22q13.2 |
| | | | GRID, GRPL, | |
| | | | GrbX, Mona, | |
| | | | GRB2L, GRBLG, | |
| | | | Grf40, GRAP-2 | |
| GRAP | 10750 | 1 | MGC64880 | 17p11.2 |
| GRB2 | 2885 | 1 | ASH, Grb3-3, | 17q24–q25 |
| | | | EGFRBP-GRB2 | |
| GRB7 | 2886 | 1 | | 17q12 |
| GRB10 | 2887 | 1 | RSS, IRBP, | 7p12–p11.2 |
| | | | MEG1, GRB-IR, | |
| | | | KIAA0207 | |
| GRB14 | 2888 | 1 | | 2q22–q24 |
| HCK | 3055 | 1 | JTK9 | 20q11–q12 |
| HSH2D | 84941 | 1 | FLJ14886, HSH2, | 19p13.11 |
| | | | ALX | |
| ITK | 3702 | 1 | EMT, LYK, | 5q31–q32 |
| | | | PSCTK2 | |
| JAK1 | 3716 | 1 | JAK1A, JAK1B, | 1p32.3–p31.3 |
| | | | JAK-1 | |
| JAK2 | 3717 | 1 | JAK-2 | 9p24 |
| JAK3 | 3718 | 1 | JAK-3, JAKL, | 19p13.1 |
| | | | LJAK | |
| LCK | 3932 | 1 | LSK | 1p34.3 |
| LNK | 10019 | 1 | | 12q24 |
| LYN | 4067 | 1 | JTK8 | 8q13 |
| MATK | 4145 | 1 | LSK, CHK, CTK, | 19p13.3 |
| | | | HYL, HHYLTK, | |
| | | | MGC1708, | |
| | | | MGC2101, | |
| | | | DKFZp434N1212 | |
| MIST | 116449 | 1 | CLNK | 4p16.1 |
| NCK1 | 4690 | 1 | NCK, NCKalpha, | 3q21 |
| | | | MGC12668 | |
| NCK2 | 8440 | 1 | GRB4, NCKbeta | 2q12 |
| PIK3R1 | 5295 | 2 | P85A, PIK3R1, | 5q13.1 |
| | | | GRB1 | |
| PIK3R2 | 5296 | 2 | P85B, PIK3R2 | 19q13.2–q13.4 |
| PIK3R3 | 8503 | 2 | P85G, PIK3R3, | 1p34.1 |
| | | | p55-GAMMA | |
| PLCγ1 | 5335 | 2 | PLC1, PLC148, | 20q12–q13.1 |
| | | | PLC-II | |
| PLCγ2 | 5336 | 2 | | 16q24.1 |
| PTPN11 | 5781 | 2 | CFC, NS1, SHP2, | 12q24 |
| | | | BPTP3, PTP2C, | |
| | | | PTP-1D, SH- | |
| | | | PTP2, SH-PTP3, | |
| | | | MGC14433, | |
| | | | PRO1847 | |
| PTPN6 | 5777 | 2 | HCP, HCPH, | 12p13 |
| | | | SHP-1, HPTP1C, | |
| | | | PTP-1C, SHP-1L, | |
| | | | SH-PTP1 | |
| RASA1 | 5921 | 2 | RASA, GAP, | 5q13.3 |
| | | | RASA, RASGAP, | |
| | | | p120GAP, | |
| | | | PKWS, CMAVM | |
| RIN1 | 9610 | 1 | | 11q13.2 |
| RIN2 | 54453 | 1 | RASSF4 | 20p11.22 |
| RIN3 | 79890 | 1 | FLJ11700, | 14q32.12 |
| | | | FLJ22439 | |
| SH2B | 25970 | 1 | SH2-B, | 16p11.2 |
| | | | DKFZP547G1110 | |
| SH2D1A | 4068 | 1 | DSHP, LYP, | Xq25–q26 |
| | | | SAP, XLP, | |
| | | | EBVS, IMD5, | |
| | | | XLPD, MTCP1 | |
| SH2D1B | 117157 | 1 | EAT2, EAT-2 | 1q21 |
| SH2D1C | 0 | 1 | N/A | N/A |
| SH2D2A | 9047 | 1 | VRAP, TSAD, | 1q21 |
| | | | TSAd, F2771 | |
| SH2D3A | 10045 | 1 | NSP1 | 19p13.3 |
| SH2D3C | 10044 | 1 | NSP3, CHAT | 9q34.11 |
| SH2D4A | 63898 | 1 | SH2A, FLJ20967 | 8p21.2 |
| SH2D4B | 387694 | 1 | CAI14998 | 10q22.3 |
| SH2D5 | 400745 | 1 | LOC400745 | 1p36.12 |
| SH3BP2 | 6452 | 1 | CRPM, RES4-23, | 4p16.3 |
| | | | CRBM | |
| SHB | 6461 | 1 | | 9p12–p11 |
| SHC1 | 6464 | 1 | SHC, SHCA | 1q21 |
| SHC2 | 25759 | 1 | SLI, SCK, SHC2, | 19p13.3 |
| | | | SHCB | |
| SHC3 | 53358 | 1 | NSHC, SHC3, N- | 9q22.1–q22.2 |
| | | | Shc, ShcC | |
| SHC4 | 399694 | 1 | MGC34023, | 15q21.1–q21.2 |
| | | | RaLP, ShcD | |
| SHD | 56961 | 1 | LOC56961 | 19p13.3 |
| SHE | 126669 | 1 | LOC126669 | 1q21.3 |
| SHF | 90525 | 1 | LOC90525, | 15q21.1 |
| | | | hypothetical | |
| | | | protein | |
| | | | BC007586 | |
| SHIP1 | 3635 | 1 | INPP5D, HCK, | 2q36–q37 |
| | | | MGC104855, | |
| | | | SHIP, SIP-145, | |
| | | | hp51CN | |
| SHIP2 | 3636 | 1 | INPPL1 | 11q23 |
| SLAP | 6503 | 1 | SLA1, SLAP, | 8q22.3–qter |
| | | | SLA | |
| SLAP2 | 84174 | 1 | SLA2, SLAP-2, | 20q11.23 |
| | | | FLJ21992, | |
| | | | MGC49845, | |
| | | | C20orf156 | |
| SLNK | 284948 | 1 | LOC284948, B | 2p11.2 |
| | | | cell linker protein | |
| SLP76 | 3937 | 1 | SLP-76, LCP2 | 5q33.1–qter |
| SOCS1 | 8651 | 1 | JAB, CIS1, SSI1, | 16p13.13 |
| | | | TIP3, CISH1, | |
| | | | Cish1, SSI-1, | |
| | | | SOCS-1 | |
| SOCS2 | 8835 | 1 | CIS2, SSI2, | 12q |
| | | | Cish2, SSI-2, | |
| | | | SOCS-2, STATI2 | |
| SOCS3 | 9021 | 1 | CIS3, Cish3, SSI- | 17q25.3 |
| | | | 3, SOCS-3, | |
| | | | MGC71791 | |
| SOCS4 | 122809 | 1 | SOCS7 | 14q22.2 |
| SOCS5 | 9655 | 1 | CIS6, CISH6, | 2p21 |
| | | | Cish5, SOCS-5, | |
| | | | KIAA0671 | |
| SOCS6 | 9306 | 1 | CIS4, SSI4, | 18q22.2 |
| | | | Cish4, SOCS4, | |
| | | | STAI4, STAT4, | |
| | | | STATI4, | |
| | | | HSPC060 | |
| SOCS7 | 30837 | 1 | NAP4 | 17q12 |
| SRC | 6714 | 1 | ASV, SRC1, c- | 20q12–q13 |
| | | | src, p60-Src | |
| SRMS | 6725 | 1 | SRM, C20orf148, | 20q13.33 |
| | | | dJ697K14.1 | |
| STAT1 | 6772 | 1 | ISGF-3, STAT91 | 2q32.2 |
| STAT2 | 6773 | 1 | P113, ISGF-3, | 12q13.13 |
| | | | STAT113, | |
| | | | MGC59816 | |
| STAT3 | 6774 | 1 | APRF, FLJ20882, | 17q21.31 |
| | | | MGC16063 | |
| STAT4 | 6775 | 1 | | 2q32.2–q32.3 |
| STAT5A | 6776 | 1 | MGF, STAT5 | 17q11.2 |
| STAT5B | 6777 | 1 | STAT5 | 17q11.2 |
| STAT6 | 6778 | 1 | STAT6B, | 12q13 |
| | | | STAT6C, | |
| | | | D12S1644, IL-4- | |
| | | | STAT | |
| SUPT6H | 6830 | 1 | SPT6, SPT6H, | 17q11.2 |
| | | | emb-5, | |
| | | | KIAA0162 | |
| SYK | 6850 | 2 | | 9q22 |
| TEC | 7006 | 1 | PSCTK4 | 4p12 |
| TENC1 | 23371 | 1 | C1-TEN, | 12q13.13 |
| | | | KIAA1075, | |
| | | | C1TEN | |
| TNS1 | 7145 | 1 | PRO0929, | 2q35–q36 |
| | | | FLJ10923, | |
| | | | DKFZP434G162, | |
| | | | TNS | |
| TNS3 | 64759 | 1 | TEM6, | 7p13–12.3 |
| | | | FLJ13732, | |
| | | | TENS1, | |
| | | | H_NH04I23.2 | |
| TNS4 | 84951 | 1 | CTEN, FLJ14950 | 17q21.2 |
| TXK | 7294 | 1 | RLK, TKL, | 4p12 |
| | | | BTKL, PSCTK5, | |
| | | | PTK4 | |
| TYK2 | 7297 | 1 | JTK1 | 19p13.2 |
| VAV1 | 7409 | 1 | VAV-1, Vav | 19p13.2 |
| VAV2 | 7410 | 1 | VAV-2 | 9q34.1 |
| VAV3 | 10451 | 1 | VAV-3 | 1p13.3 |
| YES | 7525 | 1 | Yes, C-YES, c- | 18p11.31–p11.21 |
| | | | yes, P61-YES, | |
| | | | HST441, YES1 | |
| ZAP70 | 7535 | 2 | SRK, STD, ZAP- | 2q12 |
| | | | 70, TZK | |

All references cited herein are hereby expressly incorporated in their entirety by reference. This includes patents, articles, applications, web sites and the like. The following non limiting examples serve to illustrate the invention.

### EXAMPLE 1

Itk mediated phosphorylation of Y180 in Itk, Y783 in PLCγ1; Btk mediated phosphorylation of Y223 in Btk; Tec mediated phosphorylation of Y188 in Tec—SH2 mediated substrate docking interactions.

During T cell signaling, Itk selectively phosphorylates a tyrosine within its own SH3 domain and a tyrosine within PLCγ1. We find that the remote SH2 domain in each of these substrates is required to achieve efficient tyrosine phosphorylation by Itk and extend this observation to two other Tec family kinases, Btk and Tec. Additionally, we detect a stable interaction between the substrate SH2 domains and the kinase domain of Itk, and find that addition of specific, exogenous SH2 domains to the in vitro kinase assay competes directly with substrate phosphorylation. Based on these results, we show that the kinetic parameters of a generic peptide substrate of Itk are significantly improved by fusing the peptide substrate to the SH2 domain of PLCγ1. This work is the first characterization of a substrate docking mechanism for the Tec kinases and provides evidence for a novel, phosphotyrosine-independent regulatory role for the ubiquitous SH2 domain.

Transfer of the γ-phosphate group of ATP to amino acid side chains is a primary mechanism of cellular signal transduction and is carried out by a large family of enzymes termed the protein kinases. The manner in which substrate recognition is achieved by the myriad of protein kinases is not completely understood but it is clear that specificity determinants can be outside of the motif immediately surrounding a particular phosphorylation site. Docking sites have been characterized for a number of protein kinase families that include JNKs, cyclin CDKs, and MAP kinases (1-10). For the subfamily of protein tyrosine kinases, the molecular determinants of substrate recognition by the C-terminal Src kinase (Csk) have been elucidated (11, 12). For the Csk tyrosine kinase, six amino acids within the large lobe of the kinase domain comprise a remote substrate-docking motif (12) that binds to a complementary surface on the substrate (11). This docking mechanism allows Csk to recognize and phosphorylate its substrate in a specific manner.

The extent to which other families of non-receptor protein tyrosine kinases use remote docking mechanisms to achieve substrate specificity is not known. Of interest here are the Tec family kinases; immunological enzymes that comprise the second largest family of non-receptor tyrosine kinases. The Tec family kinases include Itk, Btk, Tec, Rlk and Bmx (13), and each contains a Src homology 3 (SH3) domain, Src homology 2 (SH2) domain and the catalytic domain.

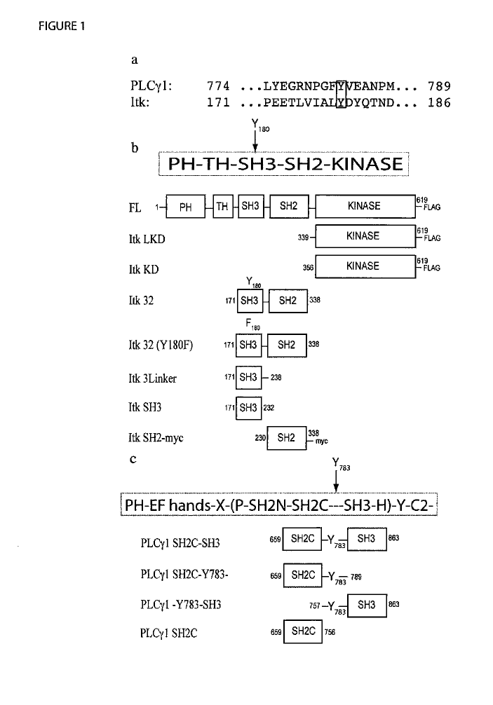

The current example focuses on Itk (Interleukin-2 tyrosine kinase), the Tec kinase that participates in signaling processes following T cell receptor engagement by phosphorylating Tyr 783 of phospholipase Cγ1 (PLCγ1) (14-19). Phosphorylation of PLCγ1 on specific tyrosine residues including Tyr 783 leads to activation of lipase activity (20). In addition to the PLCγ1 substrate, Itk also undergoes autophosphorylation on Tyr 180 within its SH3 domain (18, 21). The local amino acid sequences surrounding these two phosphorylation sites are shown in FIG. 1a and reveal little sequence similarity. Moreover, the structural context of these target tyrosines differ since Tyr 180 of Itk is embedded within the SH3 domain fold (FIG. 1b) while Tyr 783 of PLCγ1 resides in a linker region between the carboxy-terminal SH2 domain (SH2C) and SH3 domain of PLCγ1 (FIG. 1c). Thus, sequence and structural differences between two known substrates of the Itk kinase raise questions related to the mechanisms by which Itk recognizes its targets in a sufficiently specific manner to maintain the fidelity of signal transduction.

In the present example, we find that the SH2 domain within each substrate serves a docking role for recognition and phosphorylation by Itk. In each of the substrates, the SH2 domain is remote from the site of phosphorylation and shuttles the substrate to the Itk kinase domain by enhancing the affinity of the enzyme/substrate complex. We also demonstrate that efficient substrate phosphorylation by two other Tec kinases, Tec and Btk, is equally dependent on SH2 docking indicating that this mechanism is conserved across the Tec family. Moreover, in contrast to the canonical, phosphotyrosine dependent binding behavior of SH2 domains, we show that the SH2 domains of Itk substrates perform this docking function in a phosphotyrosine-independent manner.

### Results

### Efficient Phosphorylation by the Tec Kinases Requires the SH2 Domain of the Substrate.

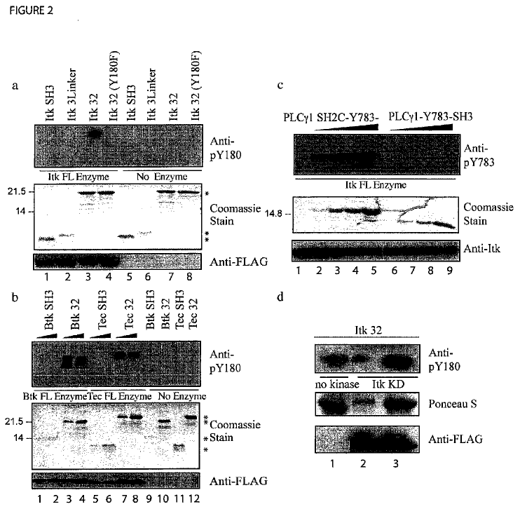

In vitro kinase assays using a panel of different substrates (FIG. 1b & c) were carried out to assess the requirements for substrate phosphorylation by the Tec kinases. Tyrosine 180 within the Itk SH3 domain is the site of Itk autophosphorylation (18) yet the isolated SH3 domain does not serve as a substrate for full length Itk (FIG. 2a, lane 1). In a similar manner, the SH3 domains of Tec and Btk (containing the autophosphorylation sites Y187 and Y223, respectively) are not phosphorylated by the Tec and Btk kinases (FIG. 2b, lanes 1, 2, 5 & 6). Finally, a fragment of PLCγ1 that contains Tyr 783 and the adjacent SH3 domain of PLCγ1 is not phosphorylated by full length Itk (FIG. 2c, lanes 6-9). Thus, efficient phosphorylation of these Tec family substrates appears to require residues outside of the target phosphorylation site.

To further test the determinants for substrate phosphorylation we created additional Itk, Tec, Btk and PLCγ1 substrate constructs and subjected them to phosphorylation by full length Itk, Tec or Btk (FIG. 2a (lanes 2-4); 2b (lanes 3, 4, 7 & 8); 2c (lanes 2-5)). In every case, only substrates that contain both the site of phosphorylation (Y180 for Itk, Y187 for Tec, Y223 for Btk, and Y783 for PLCγ1) and the adjacent SH2 domain are phosphorylated by the Tec kinases. These data indicate that the SH2 domain adjacent to each phosphorylation site is required for efficient recognition and phosphorylation by the full-length Tec kinases. To test whether substrate phosphorylation can be achieved by the catalytic domain alone, the Itk SH3-SH2 substrate (Itk 32) was subjected to phosphorylation by the isolated Itk kinase domain (Itk KD). The Itk catalytic fragment leads to substrate phosphorylation (FIG. 2d) suggesting that the kinase domain of Itk is sufficient for recognition of the SH2 domain-containing substrates.

The SH2 domains of Itk and PLCγ1 interact with the Itk kinase domain in a phosphotyrosine-independent manner.

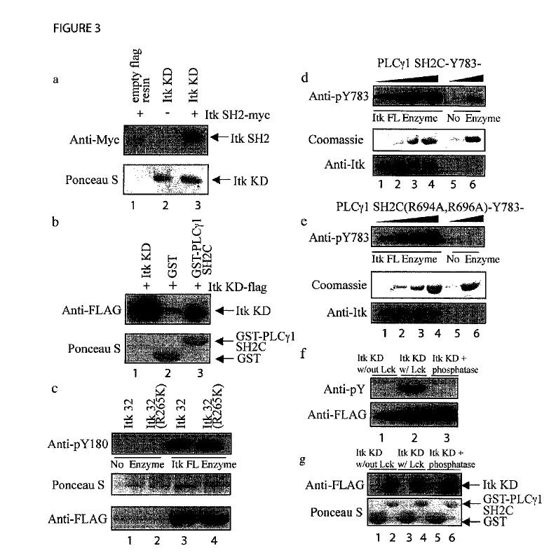

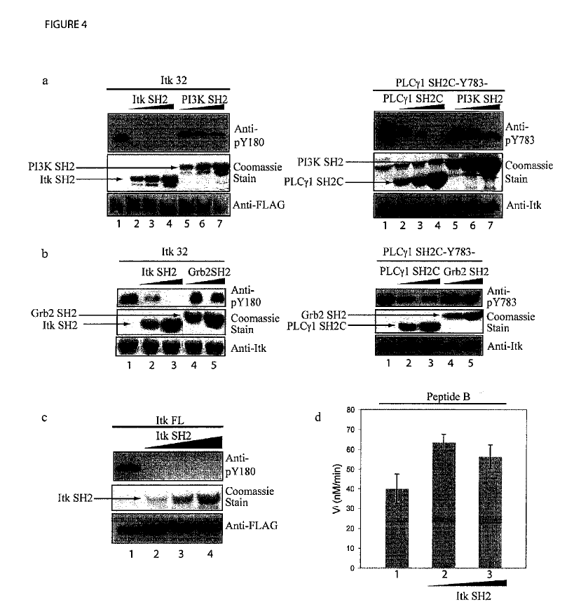

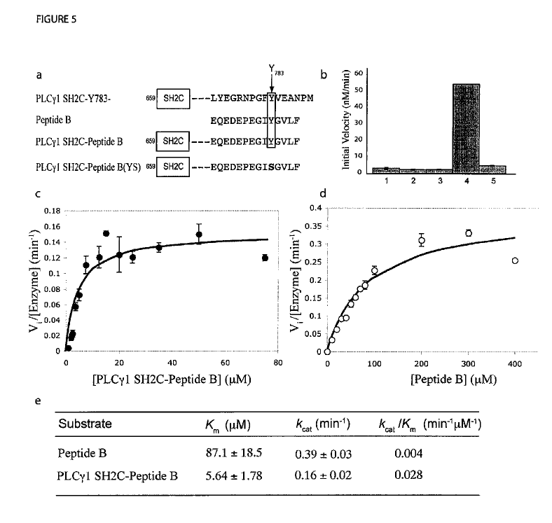

Given the observation that the Itk kinase domain alone can phosphorylate substrate and that each substrate requires the presence of the SH2 domain adjacent to the target tyrosine residue, we investigated the extent to which the isolated SH2 domains of Itk and PLCγ1 interact directly with the Itk kinase domain. Myc-tagged Itk SH2 domain, GST-tagged PLCγ1 C-terminal SH2 (PLCγ1 SH2C) domain and FLAG-tagged Itk kinase domain were purified and subjected to pull-down experiments. The Itk kinase domain interacts directly with both the Itk SH2 domain and the PLCγ1 SH2C domain (FIG. 3 a,b). We tested the nature of these interactions by mutating R265 in the Itk SH2 domain (22, 23) and R694 and R696 in PLCγ1 SH2C domain (24) to abolish phosphotyrosine mediated interactions of these SH2 domains. Mutation of the conserved arginines in the SH2 domains of both substrates has no discernable effect on substrate recognition and phosphorylation (FIG. 3c,d & e). To further probe phosphotyrosine requirements, we tested the effect of varying the phosphorylation state of the Itk kinase domain on its interaction with the PLCγ1 SH2C domain. Itk kinase domain that is expressed in insect cells without co-expression of Lck does not react with anti-pY antibody (FIG. 3f (lane 1)) indicating an absence of phosphorylated tyrosine within the Itk molecule. In contrast, Itk kinase domain that is co-expressed with Lck is expected to be phosphorylated on Tyr 511 in the activation loop. Indeed, a phosphotyrosine blot of the Itk kinase domain that is co-expressed with Lck reveals robust phosphorylation (FIG. 3f (lane 2)). Treatment with alkaline phosphatase effectively dephosphorylates the Itk kinase domain as evidenced by the absence of reactivity to an anti-phosphotyrosine antibody (FIG. 3f (lane 3)). Using this panel of Itk kinase domain preparations we found that PLCγ1 SH2C domain binds to the Itk kinase domain regardless of the phosphorylation status of the Itk kinase domain (FIG. 3g). Thus, several pieces of evidence point to a phosphotyrosine-independent interaction between the Itk kinase domain and the SH2 domains of Itk and PLCγ1 that mediates recognition and phosphorylation of these physiological substrates.

Free SH2 Domain Competes with SH2 Domain-Containing Substrate and Reduces Phosphorylation of the Substrate.

If the Itk SH2 domain and PLCγ1 SH2C domain are indeed docking sites required for the phosphorylation of Itk substrates, we expect that exogenous SH2 domain (either Itk SH2 or PLCγ1 SH2C) should effectively compete for the binding site on the Itk kinase domain and inhibit phosphorylation of the substrates. To address this, phosphorylation of Itk 32 and PLCγ1 SH2C-Y783- was monitored in the presence of increasing concentrations of either free Itk SH2 domain or free PLCγ1 SH2C domain (FIG. 4a,b). For both substrates, the corresponding free SH2 domain inhibits substrate phosphorylation (FIG. 4a,b) while two different control SH2 domains (derived from PI3K and Grb2) do not inhibit the phosphorylation of the substrates even at large molar excess (FIG. 4a,b). Inhibition by exogenous SH2 domain extends to phosphorylation of the full-length protein. In this case, Tyr 180 within full length Itk is the substrate and autophosphorylation at this site is greatly diminished upon addition of free Itk SH2 domain (FIG. 4c).

Finally, we tested whether addition of free SH2 domain to the in vitro kinase assay affects the phosphorylation of peptide B; a small model peptide substrate used previously to measure Itk activity in vitro (25). The levels of peptide B phosphorylation as indicated by initial velocity measurements do not change significantly with increasing concentrations of Itk SH2 domain (FIG. 4d) suggesting that SH2 binding to the kinase domain does not occur in a manner that directly interferes with peptide B binding to the active site. Furthermore, this result points to the absence of toxicity effects on Itk catalytic activity upon addition of free SH2 domain providing further support for the direct competition with substrate shown in FIG. 4a-c. Thus, a specific interaction between the SH2 domain of the Itk protein substrates and the Itk kinase domain is required for efficient substrate phosphorylation and appears to be localized outside of the catalytic cleft.

### SH2 Domain Enhances Substrate-Binding Affinity of a Generic Substrate.

The data described above predict that the SH2 domain within the substrates of the Tec kinases serves a docking role and likely increases the affinity of substrates for the catalytic domain. To directly test this hypothesis, the PLCγ1 SH2C domain was covalently linked to the amino terminus of Peptide B (FIG. 5a). To ensure that the desired tyrosine residue is the only site within this construct that undergoes phosphorylation, we constructed a PLCγ1 SH2C-Peptide B mutant that replaced the putative site of tyrosine phosphorylation with serine (FIG. 5a). This mutant did not incorporate phosphate indicating that the tyrosine within the Peptide B sequence is the only site in the PLCγ1 SH2C-Peptide B fusion that is phosphorylated by Itk (FIG. 5b).

Quantitative kinetic constants were compared for Peptide B alone and the PLCγ1 SH2-Peptide B fusion (FIG. 5c-e). Covalent attachment of the PLCγ1 SH2C domain to Peptide B increases substrate affinity as indicated by a reduced Kₘ value; 5.64 μM for the SH2-Peptide B fusion compared to 87 μM for Peptide B alone. The k_{cat} value only changed to a small extent suggesting that the SH2 domain primarily serves as a docking site to facilitate substrate recognition and binding by the Itk kinase domain. Thus, when linked to the PLCγ1 SH2C domain, Peptide B is a better substrate for Itk (exhibiting a 15-fold increase in substrate affinity) providing further support for the finding that Itk catalytic efficiency and selectively depends upon docking interactions with the substrate SH2 domain.

### Discussion

We have demonstrated a previously unrecognized docking role for the SH2 domain within the substrates of the Tec kinases that facilitates phosphorylation at a remote tyrosine. Many kinase sequences contain SH2 domains and these binding modules are known to affect the association of the parent kinase molecule within a signaling complex (26). Additionally, processive phosphorylation mediated by SH2/phosphotyrosine interactions has been described previously (27-29). In those examples, a tyrosine kinase creates a binding site on the substrate for its own internal SH2 domain; the ensuing SH2-substrate interaction leads to efficient phosphorylation at additional sites on the substrate. In contrast to the role of the SH2 domain in the processive phosphorylation mechanism, the results presented here suggest that SH2 domains within the substrates of the Tec kinases harbor a recognition motif that is required for efficient and selective phosphorylation of the target tyrosine. For example, the direct substrate of Itk in T cells, PLCγ1, harbors an SH2 domain (SH2C) that binds directly to the Itk kinase domain allowing the downstream PLCγ1 tyrosine (Tyr 783) to be efficiently phosphorylated by Itk. As well, autophosphorylation of Try 180 in the Itk SH3 domain depends on Itk SH2 mediated interactions with the Itk kinase domain. This mode of substrate recognition is evident within the full-length enzyme as addition of competing exogenous Itk SH2 domain to full length Itk completely eliminates Itk autophosphorylation (see FIG. 4c). To our knowledge, this direct kinase domain—SH2 interaction is a novel mode of substrate recognition that provides significant insight into how the Tec kinases achieve fidelity in their interactions with appropriate substrates and avoid deleterious ‘cross-talk’ with other substrates.

Our findings are consistent with a previous study of autophosphorylation within the Tec family kinases (21) that provided a hint into the docking role of the SH2 domain. Smith and co-workers qualitatively showed that the SH3-S2 fragments of Btk and Itk are both phosphorylated preferentially to the isolated Btk SH3 domain by the Btk kinase (21). The authors speculated that the SH3-SH2 substrate may have additional interactions with the kinase or that the site of phosphorylation in the SH3 domain bercomes more accessible in the larger SH3-SH2 substrate. They also left open the possibility that there are new phosphorylation sites on the larger SH3-SH2 substrate but report that the SH2 domain of Btk is not a substrate for Btk. The data we present strongly supports a model where the SH2 domain serves a direct docking role that is a significant determinant of substrate specificity for the Tec kinases. It is especially interesting to note that Smith and co-workers report phosphorylation levels for the SH3-SH2 fragment that are 5- to 8-fold higher than the isolated SH3 domain (21). This is in excellent agreement with kinetic data shown in FIG. 5 that indicate that Peptide B is a better substrate by 7-fold (k_{cat}/Km) when tethered to the SH2 domain of PLCγ1. Finally, our use of phosphotyrosine specific antibodies and mutation of the target tyrosine supports the notion that additional phosphorylation sites in the SH2 domain containing substrates are not leading to the observed increase in substrate phosphorylation (21).

The phosphotyrosine-independent nature of the binding event between Itk kinase domain and SH2 domains, highlights alternative means by which SH2 domains can engage their targets. To date, targeting the conserved arginines in the phosphotyrosine binding pocket of the SH2 domain has become the traditional ‘loss-of-function’ mutation for this binding module. A full appreciation of phosphotyrosine-independent SH2 binding will lead to revised models of signaling complexes. For PLCγ1 signaling in particular, traditional mutagenic approaches targeting the phosphotyrosine binding site of the SH2C domain have previously led to conclusions that this portion of PLCγ1 is dispensable for PLCγ1 recruitment and phosphorylation (30, 31). More recently, the extended experimental approach of Samelson and co-workers showed that all three SH domains including SH2C are required for phosphorylation of PLCγ1 in T cells (32) yet these studies still relied on the mutation of arginine in the phosphotyrosine binding pocket of the SH2C domain. Certainly, the phosphotyrosine-independent docking role for the PLCγ1 SH2C domain would have been missed by standard ‘loss-of-function’ mutations in the SH2 domain.

To fully characterize this novel, SH2 dependent substrate recognition mechanism, the interaction sites on both the Itk kinase domain and the substrate SH2 domains must be mapped at the molecular level. In addition to providing insight into the PLCγ1 activation mechanism, the precise arrangement of the Itk SH2 domain within the full-length Itk enzyme is of particular interest. In other tyrosine kinases, the non-catalytic SH2 domain plays a defined role in regulating kinase activity by forming direct intramolecular contacts to the small lobe of the kinase domain (33, 34). For Itk we have also found that the SH2 domain positively contributes to the regulation of catalytic activity (Joseph and Andreotti, unpublished results). Thus, the emerging bifunctional nature of the Itk SH2 domain (as substrate recognition module and regulatory domain) raises interesting questions related to how the Itk SH2 domain orchestrates its various roles during enzymatic catalysis. Does this SH2 domain interact with its neighboring kinase domain in a single mode that achieves both regulation of kinase activity and substrate recognition of the autophosphorylation site or does the SH2 domain shift between multiple interaction sites on the kinase domain to achieve these functions? With molecular level details still forthcoming, it is nevertheless clear that the data we present here point to a specific SH2 domain mediated docking mechanism by which the Tec kinases recognize and phosphorylate their substrates. One extension of this result is that screening for interactions between the Tec kinase domains and unrelated SH2 domains may provide leads to identify additional substrates for this important family of tyrosine kinases. In addition, we have shown that Itk substrates can be displaced from the active site by addition of exogenous SH2 domain. This result promises an exciting strategy to attenuate Itk mediated signaling that may have significant selectivity advantages since it would not require the development of molecules that discriminate between very similar kinase active sites.

### Materials and Methods

Baculoviral and bacterial constructs—Full-length Itk, Btk and Tec and the Itk kinase domain fragments were PCR amplified using a reverse primer that encoded a FLAG epitope tag. The PCR products were cloned into the pENTR/D-TOPO vector (Invitrogen) by TOPO cloning. All of the PLCγ1, Itk, Btk and Tec fragments that do not contain kinase domain were subcloned into the pGEX-2T expression vector (GE Healthcare) for production and purification from bacteria as described previously (35). The N-terminal Phosphatidylinositol 3-Kinase (p85α) SH2 domain (PI3K SH2) and Grb2 SH2 domain were cloned into pGEX-5X-1 by PCR. The pGEX-2T PLCγ1 C-terminal SH2 domain-PeptideB fusion was created by PCR and included an N-terminal biotinylation sequence. For this construct and the pGEX-2T PLCγ1 SH2C-PeptideB(YS) mutant, Tyr 771 was mutated to Phe to avoid phosphorylation at this site during analysis of the kinetics and Tyr 775 has been deleted by replacement with the Peptide B sequence (36). Biotinylated protein was produced in BL21 cells by co-expressing Biotin ligase. The biotinylated protein was purified by affinity purification using an Avidin resin (Pierce). For the Itk substrate constructs that include both the SH3 and SH2 domain (Itk32 and Itk32(Y180F)) we also introduced two point mutants (W208K and I282A) to eliminate complications that may arise from dimerization of the substrate as reported previously (35). The wild type Itk SH3-SH2 construct is phosphorylated by Itk to the same extent as the double mutant (data not shown). All mutations were made using the site directed mutagenesis kit (Stratagene). All constructs were verified by sequencing at the Iowa State University DNA synthesis and sequencing facility.

Baculovirus production—The pENTR vectors with various inserts were recombined in vitro with BaculoDirect C-Term Linear DNA (Invitrogen) using LR Clonase II enzyme (Invitrogen). The DNA was then transfected into Sƒ9 cells using Effectene (Qiagen). Three rounds of viral selection and amplifications were carried out. For protein production, the cells were infected with a 1:1 ratio of Itk (or Btk or Tec): Lck baculovirus unless otherwise indicated. The cells were harvested 72 hrs post-infection, rinsed once with phosphate buffered saline (PBS) and stored at −80° C. Following purification each Itk enzyme construct was assessed for Tyr 511 phosphorylation using a Btk pY551 specific antibody. This step ensured that co-expression with Lck produced appropriately activated Itk (18).

Pull-Down Assays—Purified 0.5 μM FLAG-tagged Itk kinase domain (Itk KD) immobilized on an anti-FLAG-resin was incubated with 1 μM purified Myc-tagged Itk SH2 (Itk SH2-myc) domain in RIPA buffer (50 mM Tris pH7.4, 150 mM NaCl, 1 mM PMSF, 1% NP40, 1 mM EDTA, 1 mM NaF) at 4° C. overnight. The samples were washed, boiled, resolved on an SDS-PAGE gel, transferred to PVDF membrane and western blotted with an anti-Myc antibody (Invitrogen). For PLCγ1 pull downs 0.22 μM FLAG-tagged Itk kinase domain (Itk KD) was incubated with 3.8 μM of purified GST or 3.8 μM GST-PLCγ1 SH2C immobilized on glutathione beads. The samples were treated as before and western blotted using an anti-FLAG antibody. Unphosphorylated FLAG-tagged Itk Kinase domain (Itk KD) was prepared by treating 13.6 μM Itk KD with 1 unit/μL of Alkaline phosphatase (New England Biolab) for 1 hr at 37° C.

Protein purification—Purification of baculovirus produced protein was carried as previously described (25). Cell pellets were resuspended in lysis buffer (50 mM Tris pH 8.0, 500 mM NaCl, 2 mM EDTA, 1 mM PMSF) and lysed by dounce homogenization. The homogenate was spun at 16K for 1 hr at 4° C. Glycerol was added to the supernatant to a final volume of 10%, and then incubated with anti-FLAG M2 affinity resin (Sigma) for 5 hrs at 4° C. The resin was rinsed five times in wash buffer (50 mM Tris pH 8.0, 500 mM NaCl, 1 mM PMSF, 10% glycerol), and if necessary for kinase assays, the protein was eluted in elution buffer (wash buffer with 200 μg/ml FLAG peptide) and stored at −80° C. The purified protein was quantified by measuring absorbance at 280 nm. All proteins were greater than 95% pure by Coomassie staining. We note that the Btk SH3 domain runs higher than the actual molecular weight and the closely related Tec SH3 domain (FIG. 2b). The molecular weight of both Btk SH3 domain and Tec SH3 domain were confirmed by mass spectrometry (data not shown). Additionally, identical preparations of Itk mutant enzymes that are kinase inactive (K390R) do not show any activity toward any substrate (data not shown).

Kinase assays and Western Blotting—Itk Full-length (FL), linker kinase domain (Itk LKD), kinase domain (Itk KD), full-length Btk or full-length Tec were incubated with the indicated substrates in an in vitro kinase assay buffer (50 mM Hepes pH 7.0, 10 mM MgCl₂, 1 mM DTT, 1 mg/ml BSA, 1 mM Pefabloc and 200 μM ATP) for one hour at RT. The samples were boiled, separated by SDS-PAGE and transferred onto a PVDF membrane. The membranes were then blotted with either phosphotyrosine specific antibodies (anti-pY783 for PLCγ1 or pY223 Btk antibody used to detect pY180 in Itk, pY187 in Tec and pY223 in Btk (18)), an anti-phosphotyrosine antibody (4G10 from Upstate), or an anti-FLAG antibody (Sigma) and developed by standard chemiluminescence (Pierce) methods. Quantitative kinase assays (25): Kₘ determinations for Peptide B [(Aminohexanoyl biotin-EQEDEPEGIYGVLF-NH₂) (Anaspec Inc.)] and the biotinylated PLCγ1 SH2C-Peptide B fusion were carried out by incubating purified Itk LKD enzyme in reaction buffer (50 mM Hepes pH 7.0, 10 mM MgCl₂, 1 mM DTT, 1 mg/ml BSA, 1 mM Pefabloc SC (4-(2-aminoethyl)-benzenesulfonyl-flouride) and 200 μM ATP) and 5 μCi [³²P] ATP (GE Healthcare) at room temperature. Peptide B concentration was varied between 0-400 μM and PLCγ1 SH2C-Peptide B fusion concentration was varied between 0-80 μM. The enzyme concentration used for kinetic analysis was 0.9 μM. Each assay was done in duplicate. The data obtained was fitted onto the Michealis-Menten equation using GraphFit 5 software, and the kinetic parameters were obtained.

### REFERENCES

(1) Adams, P. D., Li, X., Sellers, W. R., Baker, K. B., Leng, X., Harper, J. W., Taya, Y., and Kaelin, W. G., Jr. (1999) Retinoblastoma protein contains a C-terminal motif that targets it for phosphorylation by cyclin-cdk complexes. Mol Cell Biol 19, 1068-80.
(2) Adams, P. D., Sellers, W. R., Sharma, S. K., Wu, A. D., Nalin, C. M., and Kaelin, W. G., Jr. (1996) Identification of a cyclin-cdk2 recognition motif present in substrates and p21-like cyclin-dependent kinase inhibitors. Mol Cell Biol 16, 6623-33.
(3) Chang, C. I., Xu, B. E., Akella, R., Cobb, M. H., and Goldsmith, E. J. (2002) Crystal structures of MAP kinase p38 complexed to the docking sites on its nuclear substrate MEF2A and activator MKK3b. Mol Cell 9, 1241-9.
(4) Jacobs, D., Glossip, D., Xing, H., Muslin, A. J., and Komfeld, K. (1999) Multiple docking sites on substrate proteins form a modular system that mediates recognition by ERK MAP kinase. Genes Dev 13, 163-75.
(5) Kallunki, T., Deng, T., Hibi, M., and Karin, M. (1996) c-Jun can recruit JNK to phosphorylate dimerization partners via specific docking interactions. Cell 87, 929-39.
(6) Lee, T., Hoofnagle, A. N., Kabuyama, Y., Stroud, J., Min, X., Goldsmith, E. J., Chen, L., Resing, K. A., and Ahn, N. G. (2004) Docking motif interactions in MAP kinases revealed by hydrogen exchange mass spectrometry. Mol Cell 14, 43-55.
(7) Luciani, M. G., Hutchins, J. R., Zheleva, D., and Hupp, T. R. (2000) The C-terminal regulatory domain of p53 contains a functional docking site for cyclin A. J Mol Biol 300, 503-18.
(8) Schulman, B. A., Lindstrom, D. L., and Harlow, E. (1998) Substrate recruitment to cyclin-dependent kinase 2 by a multipurpose docking site on cyclin A. Proc Natl Acad Sci USA 95, 10453-8.
(9) Tanoue, T., Maeda, R., Adachi, M., and Nishida, E. (2001) Identification of a docking groove on ERK and p38 MAP kinases that regulates the specificity of docking interactions. Embo J 20, 466-79.
(10) Wallace, M., and Ball, K. L. (2004) Docking-dependent regulation of the Rb tumor suppressor protein by Cdk4. Mol Cell Biol 24, 5606-19.
(11) Lee, S., Ayrapetov, M. K., Kemble, D. J., Parang, K., and Sun, G. (2006) Docking-based substrate recognition by the catalytic domain of a protein tyrosine kinase, C-terminal Src kinase (Csk). J Biol Chem 281, 8183-9.
(12) Lee, S., Lin, X., Nam, N. H., Parang, K., and Sun, G. (2003) Determination of the substrate-docking site of protein tyrosine kinase C-terminal Src kinase. Proc Natl Acad Sci USA 100, 14707-12.
(13) Berg, L. J., Finkelstein, L. D., Lucas, J. A., and Schwartzberg, P. L. (2005) Tec family kinases in T lymphocyte development and function. Annu Rev Immunol 23, 549-600.
(14) Liu, K. Q., Bunnell, S. C., Gurniak, C. B., and Berg, L. J. (1998) T cell receptor-initiated calcium release is uncoupled from capacitative calcium entry in Itk-deficient T cells. J Exp Med 187, 1721-7.
(15) Miller, A. T., and Berg, L. J. (2002) Defective Fas ligand expression and activation-induced cell death in the absence of IL-2-inducible T cell kinase. J Immunol 168, 2163-72.
(16) Perez-Villar, J. J., and Kanner, S. B. (1999) Regulated association between the tyrosine kinase Emt/Itk/Tsk and phospholipase-C gamma 1 in human T lymphocytes. J Immunol 163, 6435-41.
(17) Schaeffer, E. M., Debnath, J., Yap, G., McVicar, D., Liao, X. C., Littman, D. R., Sher, A., Varmus, H. E., Lenardo, M. J., and Schwartzberg, P. L. (1999) Requirement for Tec kinases Rlk and Itk in T cell receptor signaling and immunity. Science 284, 638-41.
(18) Wilcox, H. M., and Berg, L. J. (2003) Itk phosphorylation sites are required for functional activity in primary T cells. J Biol Chem 278, 37112-21.
(19) Bonvini, E., DeBell, K. E., Veri, M. C., Graham, L., Stoica, B., Laborda, J., Aman, M. J., DiBaldassarre, A., Miscia, S., and Rellahan, B. L. (2003) On the mechanism coupling phospholipase Cgammal to the B- and T-cell antigen receptors. Adv Enzyme Regul 43, 245-69.
(20) Sekiya, F., Poulin, B., Kim, Y. J., and Rhee, S. G. (2004) Mechanism of tyrosine phosphorylation and activation of phospholipase C-gamma 1. Tyrosine 783 phosphorylation is not sufficient for lipase activation. J Biol Chem 279, 32181-90.
(21) Nore, B. F., Mattsson, P. T., Antonsson, P., Backesjo, C. M., Westlund, A., Lennartsson, J., Hansson, H., Low, P., Ronnstrand, L., and Smith, C. I. (2003) Identification of phosphorylation sites within the SH3 domains of Tec family tyrosine kinases. Biochim Biophys Acta 1645, 123-32.
(22) Bunnell, S. C., Diehn, M., Yaffe, M. B., Findell, P. R., Cantley, L. C., and Berg, L. J. (2000) Biochemical interactions integrating Itk with the T cell receptor-initiated signaling cascade. J Biol Chem 275, 2219-30.
(23) Ching, K. A., Grasis, J. A., Tailor, P., Kawakami, Y., Kawakami, T., and Tsoukas, C. D. (2000) TCR/CD3-Induced activation and binding of Emt/Itk to linker of activated T cell complexes: requirement for the Src homology 2 domain. J Immunol 165, 256-62.
(24) Poulin, B., Sekiya, F., and Rhee, S. G. (2005) Intramolecular interaction between phosphorylated tyrosine-783 and the C-terminal Src homology 2 domain activates phospholipase C-gamma1. Proc Natl Acad Sci USA 102, 4276-81.
(25) Hawkins, J., and Marcy, A. (2001) Characterization of Itk tyrosine kinase: contribution of noncatalytic domains to enzymatic activity. Protein Expr Purif 22, 211-9.
(26) Pawson, T., Raina, M., and Nash, P. (2002) Interaction domains: from simple binding events to complex cellular behavior. FEBS Lett 513, 2-10.
(27) Mayer, B. J., Hirai, H., and Sakai, R. (1995) Evidence that SH2 domains promote processive phosphorylation by protein-tyrosine kinases. Curr Biol 5, 296-305.
(28) Lewis, L. A., Chung, C. D., Chen, J., Pames, J. R., Moran, M., Patel, V. P., and Miceli, M. C. (1997) The Lck SH2 phosphotyrosine binding site is critical for efficient TCR-induced processive tyrosine phosphorylation of the zeta-chain and IL-2 production. J Immunol 159, 2292-300.
(29) Pellicena, P., Stowell, K. R., and Miller, W. T. (1998) Enhanced phosphorylation of Src family kinase substrates containing SH2 domain binding sites. J Biol Chem 273, 15325-8.
(30) Irvin, B. J., Williams, B. L., Nilson, A. E., Maynor, H. O., and Abraham, R. T. (2000) Pleiotropic contributions of phospholipase C-gamma1 (PLC-gamma1) to T-cell antigen receptor-mediated signaling: reconstitution studies of a PLC-gamma1-deficient Jurkat T-cell line. Mol Cell Biol 20, 9149-61.
(31) Stoica, B., DeBell, K. E., Graham, L., Rellahan, B. L., Alava, M. A., Laborda, J., and Bonvini, E. (1998) The amino-terminal Src homology 2 domain of phospholipase C gamma 1 is essential for TCR-induced tyrosine phosphorylation of phospholipase C gamma 1. J Immunol 160, 1059-66.
(32) Braiman, A., Barda-Saad, M., Sommers, C. L., and Samelson, L. E. (2006) Recruitment and activation of PLCgamma1 in T cells: a new insight into old domains. Embo J 25, 774-84.
(33) Ogawa, A., Takayama, Y., Sakai, H., Chong, K. T., Takeuchi, S., Nakagawa, A., Nada, S., Okada, M., and Tsukihara, T. (2002) Structure of the carboxyl-terminal Src kinase, Csk. J Biol Chem 277, 14351-4.
(34) Xu, W., Harrison, S. C., and Eck, M. J. (1997) Three-dimensional structure of the tyrosine kinase c-Src. Nature 385, 595-602.
(35) Brazin, K. N., Fulton, D. B., and Andreotti, A. H. (2000) A specific intermolecular association between the regulatory domains of a Tec family kinase. J Mol Biol 302, 607-23.
(36) Serrano, C. J., Graham, L., DeBell, K., Rawat, R., Veri, M. C., Bonvini, E., Rellahan, B. L., and Reischl, I. G. (2005) A new tyrosine phosphorylation site in PLC gamma 1: the role of tyrosine 775 in immune receptor signaling. J Immunol 174, 6233-7.

### EXAMPLE 2

Lck mediated phosphorylation of Y511 in Itk—an SH2 Mediated Substrate Docking Interaction.

We have also found that the phosphorylation of Itk Y511 by the Src family Lck kinase is mediated by a kinase domain/SH2 interaction. Phosphorylation of Y511 in Itk is a requirement for activation of Itk and so disruption of this event in T cells should render Itk inactive (1).

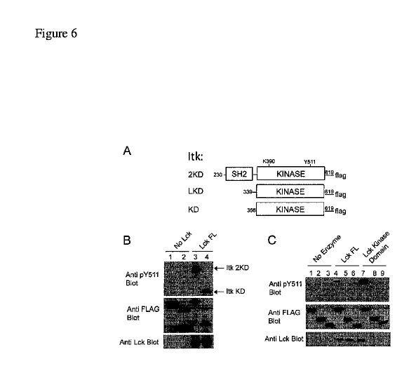

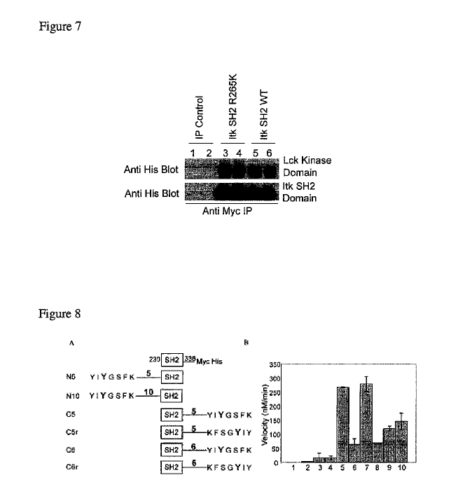

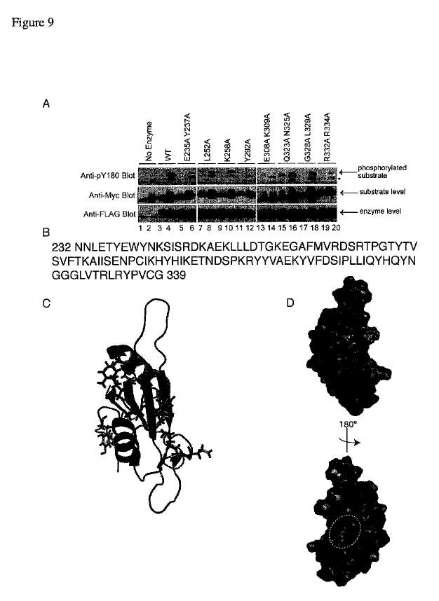

This unexpected finding is based on data that show that phosphorylation of the Itk kinase domain alone (on Y511) by Lck is very inefficient while Lck phosphorylation of the SH2-kinase fragment of Itk proceeds readily (FIG. 6). We identify a direct interaction between the Lck kinase domain and the Itk SH2 domain that mediates Lck activation of Itk (FIG. 7). The interaction is independent of the canonical phosphotyrosine binding function of the SH2 domain since mutation of R265 has no effect on binding to the Lck kinase domain (FIG. 7). Thus, the Lck kinase domain/Itk SH2 domain interaction is an extremely attractive target for discovery of small molecules that interfere with this protein-protein interaction. If Lck is unable to phosphorylate Y511 on Itk in T cells it is expected that the phenotype would mirror that of the Itk Y511F mutant described by Wilcox and Berg (1). In that work the Y511F mutant is severely defective in cytokine production.

We further explored the requirement for the Itk SH2 domain for Lck kinase mediated phosphorylation. In the experiments shown in FIG. 8 we made use of a short peptide substrate of Lck (YIYGSFK) and measured phosphorylation kinetics of this peptide free in solution and tethered to the Itk SH2 domain. Six different tethered constructs were made that differ with respect to where the peptide is attached to the Itk SH2 domain (either amino- or carboxy-terminus) and with respect to the number of amino acid residues linking the peptide to the SH2 domain. We find that the tyrosine (Y) in the peptide is most efficiently phosphorylated when attached to the carboxy-terminus of the SH2 domain and this tethered substrate is a better substrate by an order of magnitude. Thus, the data in FIGS. 6-8 all indicate that the Itk SH2 domain is a docking site for the Lck kinase enzyme.

The data in examples 1 and 2 show that inhibition of either Lck mediated phosphorylation of Itk Y511 and/or Itk mediated phosphorylation of PLCγ1 Y783 should inhibit signals emanating from the T cell receptor by shutting down Itk. The nature of the docking interaction described here means that inhibition can be achieved without direct inhibition of a kinase active site. Active site inhibitors are the common approach to modulating kinase mediated signaling but the promise of this approach is diminished due to problems associated with achieving sufficient selectivity.

### Materials and Methods:

Baculoviral and bacterial constructs—Catalytically inactive (K390R) Itk SH2-kinase domain (mouse sequence), Itk linker kinase domain or Itk kinase domain fragments were PCR amplified using a reverse primer that encoded a FLAG epitope tag. The PCR products were cloned into the pENTR/D-TOPO vector (Invitrogen) by TOPO cloning. The Itk SH2 domain was cloned into the pTrcHis2-TOPO vector (Invitrogen) such that it was in frame with the C-terminal vector derived Myc and His tag. The Itk SH2 Lck peptide substrate (YIYGSFK) fusions were cloned into the pET28a vector with an N-terminal His tag. The five amino acid linker between the peptide and the Itk SH2 domain consisted of GlyGlyGlyGlySer and repeats of the same for the ten amino acid linker. All mutations were made using the site directed mutagenesis kit (Stratagene). The bacterial expression construct for His-tagged wild type Lck kinase domain was a kind gift from Dr. John Kuriyan. The Lck full-length baculovirus was a kind gift from Dr. Leslie Berg. All constructs were verified by sequencing at the Iowa State University DNA synthesis and sequencing facility.

Baculovirus production—The pENTR vectors with various inserts were recombined in vitro with BaculoDirect C-Term Linear DNA (Invitrogen) using LR Clonase II enzyme according to the manufacturers instructions (Invitrogen). The DNA was then transfected into Sƒ9 cells using Effectene (Qiagen). Three rounds of viral selection and amplifications were carried out as described in the instruction manual (Invitrogen). For protein production, the cells were infected with a 1:1 ratio of Itk:Lck baculovirus unless otherwise indicated. The cells were harvested 72 hrs post-infection, rinsed once with phosphate buffered saline (PBS) and stored at −80° C.

Protein purification—Purification of baculovirus produced protein was carried as follows. The cell pellets were resuspended in lysis buffer (50 mM Tris pH 8.0, 500 mM NaCl, 2 mM EDTA, 1 mM PMSF) and lysed by dounce homogenization. The homogenate was spun at 16K for 1 hr at 4° C. Glycerol was added to the supernatant to a final volume of 10%, and then incubated with anti-FLAG M2 affinity resin (Sigma) for 5hrs at 4° C. The resin was rinsed five times in wash buffer (50 mM Tris pH 8.0, 500 mM NaCl, 1 mM PMSF, 10% glycerol), and the protein was eluted in elution buffer (wash buffer with 200 μg/ml FLAG peptide) and stored at −80° C. All bacterial expression was carried out in BL21(DE3) cells, expect for Lck expression where ArcticExpress (Stratagene) cells were used. Bacterially expressed proteins were purified using Ni-NTA resin (Qiagen) according to the manufacturers instructions. The proteins were eluted in elution buffer (50 mM Hepes pH 8.0, 150 mM NaCl, 250 mM imidazole) and stored at 4° C. with DTT at 2 mM final concentration. Lck was eluted with elution buffer containing 10% glycerol and was stored at −80° C. The purified protein was quantified by measuring absorbance at 280 nm. All proteins were greater than 95% pure by Coomassie staining.

In vivo kinase assay and western blotting: Sƒ9 cells were plated at a density of 4×10⁵ cells/well in a 6-well plate. The next day the cells were infected with the respective virus/es. Three days post infection, the cells were harvested, rinsed once with PBS, and lysed in radioimmunoprecipitation (RIPA) buffer (50 mM Tris pH 7.4, 150 mM NaCl, 1 mM EDTA, 1% NP-40, 1 mM NaF, 1 mM DTT, 1 mM PMSF). The lysate was sonicated briefly and spun at 10,000 g for 10min at 4° C. The supernatants were normalized for protein content using Bradford assay, separated by SDS-PAGE and transferred onto a PVDF membrane. The membranes were then blotted with a Btk phosphoTyr 551 antibody (which has been previously used to detect Itk phosphorylation on Tyr 511) or an anti-FLAG antibody (Sigma) and developed by standard chemiluminescence (Pierce) methods.

In vitro kinase assays—Lck peptide (YIYGSFK) substrates were obtained from AnaSpec. Lck kinase domain alone or protein A beads with full-length Lck immunoprecipitated from Sƒ9 cells using an anti-Lck antibody (3A5 from Santa Cruz Biotechnology) were incubated with the 1 μM various Itk substrates in an in vitro kinase assay buffer (50 mM Hepes pH 7.0, 10 mM MgCl₂, 1 mM DTT, 1 mg/ml BSA, 1 mM Pefabloc and 200 μM ATP) for one hour at RT. The samples were boiled, separated by SDS-PAGE and transferred onto a PVDF membrane. The membranes were then blotted with a Btk phosphoTyr 551 antibody or an anti-FLAG antibody (Sigma) as before. Quantitative kinase assays were carried out by incubating bacterially purified Lck kinase domain with 10 μM of various protein substrates and 400 μM of peptide substrates in reaction buffer (50 mM Hepes pH 7.0, 10 mM MgCl₂, 1 mM DTT, 1 mg/ml BSA, 1 mM Pefabloc SC (4-(2-aminoethyl)-benzenesulfonyl-fluoride) and 200 μM ATP) and 5 μCi [³²P] ATP (Amersham Biosciences) at room temperature. The reactions were stopped with 20 mM EDTA or 8M guanidine hydrochloride and spotted onto Streptavidin membranes (Pierce) to capture the biotinylated peptide substrate or P81 membranes (Whatman) to capture all the other substrates. The membranes were washed, air dried and counted by liquid scintillation counting on a Packard 1600 TR liquid scintillation counter. Each assay was done in duplicate.

Pull-Down Assays—Purified His-tagged (wild type or kinase inactive (K273R)) Lck kinase domain was incubated with 0.8 μM purified Myc-tagged Itk (wild type or R265K mutant) SH2 domain in RIPA buffer (50 mM Tris pH7.4, 150 mM NaCl, 1 mM PMSF, 1% NP40, 1 mM EDTA, 1 mM NaF) with an anti-Myc antibody and protein A beads at 4° C. overnight. The samples were washed, boiled, resolved on an SDS-PAGE gel, transferred to PVDF membrane and western blotted with an anti-His antibody (clone HIS.H8 from Upstate).
(1) Wilcox, H. M., and Berg, L. J. (2003) Itk phosphorylation sites are required for functional activity in primary T cells. J Biol Chem 278, 37112-21.
(2) Hawkins, J., and Marcy, A. (2001) Characterization of Itk tyrosine kinase: contribution of noncatalytic domains to enzymatic activity. Protein Expr Purif 22, 211-9.

### EXAMPLE 3

In examples 1 and 2 we have established that the Itk and Lck kinase domains each recognize and bind to their substrate proteins via a remote docking interaction with an SH2 domain within the substrate. The three interactions described to date are: Itk kinase domain:Itk SH2 domain (mediates phosphorylation of Y180 in Itk); Itk kinase domain:PLCγ1 SH2 domain (mediates phosphorylation of Y783 in PLCγ1) and Lck kinase domain:Itk SH2 domain (mediates phosphorylation of Y511 in Itk).

The data provided here (FIG. 9) map the molecular determinants of the Itk SH2 domain that are responsible for binding to the Itk kinase domain (interaction mediating Y180 phosphorylation). Based on a sequence alignment of the Itk SH2 domain with the other Tec kinases and two SH2 sequences that do not bind the Itk kinase domain (PI3K and Grb2) we identified surface residues that are conserved within the Tec kinases but are different in PI3K and Grb2. These SH2 surface residues were then mutated to alanine in the context of the Itk SH3-SH2 dual domain substrate (Y180 resides in the SH3 domain). We asked the question whether specific point mutations in the SH2 domain of the SH3-SH2 substrate would affect phosphorylation of Y180 in the SH3 domain of the SH3-SH2 substrate by Itk. Each mutant was compared to the wild type Itk SH3-SH2 substrate for its ability to be phosphorylated by the Itk kinase. As shown in FIG. 9A, the mutants varied in the extent to which they are phosphorylated on Y180 by Itk. Mutation of two sites (Q323, N325) had no effect on SH3-SH2 substrate phosphorylation compared to wild type substrate indicating that these residues do not contribute to the docking interaction between SH2 and kinase domains (wild type substrate is in lanes 3 & 4, mutants in lanes 15, 16; FIG., 9A). Mutation of E235, Y237, L252, K258, Y292, E308, K309, G328, L329, R332 and R334 leads to a decrease in Y180 phosphorylation indicating that these residues are important for the Itk kinase:SH2 docking interaction. Notably, mutation of a subset of these sites (E235, Y237, Y292, R332, R334) almost completely abolishes Y180 phosphorylation in the SH3-SH2 substrate (lanes 5, 6, 11, 12, 19, 20; FIG. 9A). Mutation of the others (L252, K258, E308, K309, G328, L329) partially diminishes the docking interaction as indicated by fractional phosphorylation of Y180 compared to wild type SH3-SH2 substrate (lanes 7, 8, 9, 10, 13, 14, 17, 18; FIG. 1A). Thus, these eleven residues (FIG. 9B) likely form the surface of the Itk SH2 domain that interacts with the kinase domain to mediate substrate phosphorylation.

Mapping these eleven residues on the structure of the Itk SH2 domain reveals an extended and contiguous surface (FIGS. 9C&D). This surface is on the opposite face from that which mediates canonical binding to phosphotyrosine containing ligands (FIG. 9D). Given the observation that other Tec family kinase members make use of the same kinase:SH2 docking interaction for phosphorylation of the tyrosine in their respective SH3 domains and that the indicated residues in Itk (FIG. 9B) are largely conserved among all Tec kinases, it is expected that this docking surface on the Itk SH2 domain is conserved among the five Tec kinases.

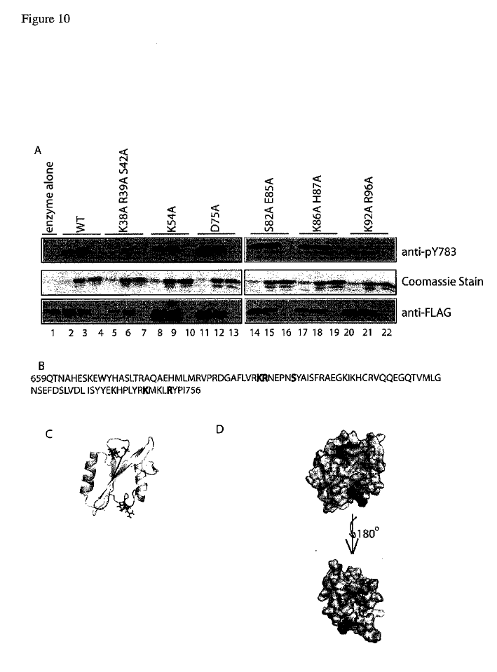

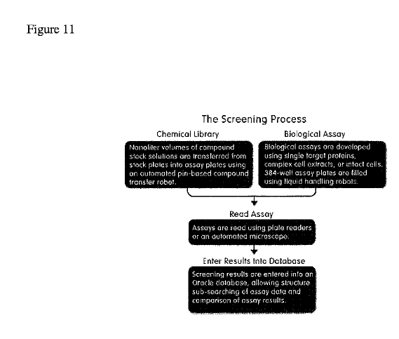

Further data are provided in FIGS. 10A-D that begin to map the molecular determinants of the PLCγ1 SH2 domain responsible for binding to the Itk kinase domain (interaction mediating Y783 phosphorylation in PLCγ1). Based on a sequence alignment of the PLCγ1 SH2 domain from a number of different species as well as two SH2 sequences that do not bind the Itk kinase domain (PI3K and Grb2) we identified surface residues that are conserved within the PLCγ1 SH2 domain but are different in PI3K and Grb2. These SH2 surface residues were then mutated to alanine in the context of the PLCγ1 SH2 linker substrate (Y783 resides in the linker between the PLCγ1 SH2 and SH3 domains). We asked the question whether specific point mutations in the SH2 domain of the PLCγ1 SH2 linker substrate affect phosphorylation of Y783 by Itk. Each mutant was compared to the wild type PLCγ1 SH2 linker substrate for its ability to be phosphorylated by the Itk kinase. As shown in FIG. 10A, mutation of K695, R696, S699, K749 and R753 leads to a significant decrease of phosphorylation of Y783 when compared to the wild type substrate indicating that these residues are critical for the PLCγ1 SH2 and Itk kinase domain interaction. Mutation of K711, D732, S739, E742, E743, H744 had no effect on PLCγ1 SH2 linker substrate phosphorylation compared to wild type substrate indicating that these residues do not contribute to the docking interaction between PLCγ1 c-terminal SH2 domain and kinase domains. Thus, these five residues likely form part of the surface of the PLCγ1 SH2 domain that interacts with the kinase domain to mediate substrate phosphorylation.

### EXAMPLE 4

### Summary

Cellular signal transduction is the process by which cells go about growing, dividing, proliferating and dying in response to specific external signals. In a healthy cell, these activities are precisely controlled in large part due to the enzymatic activity of the protein kinases. Protein kinases are an abundant class of enzymes that chemically modify other proteins by attaching a phosphate group. By inducing very specific phosphorylation cascades, protein kinases transmit most of the signals that allow cells to respond to environmental cues. In the context of disease states, the protein kinases are key targets to controlling cellular signaling and thus the progression of disease. To this end, tremendous efforts have focused on finding small molecule inhibitors of the protein kinases to serve as therapeutic agents. Inhibitors targeted toward the active site of the protein kinases have been disappointingly non-specific; causing extensive cross reactivity and unwanted side effects.

We have discovered a completely novel docking mechanism by which a specific protein kinase of the immune system recognizes and phosphorylates its substrate. We have also shown that if the docking interaction is disrupted, phosphorylation by the kinase is ceased. The remote docking mechanism that we have described means that a completely different approach can be taken toward identification of small molecules that will modulate the function of this kinase. We propose here an experimental screen for such small molecules with the expectation that lead compounds will emerge that will have the potential to be useful therapeutics. Given the novelty of this kinase docking mechanism the approach described here should provide a significantly more specific avenue toward kinase inhibitors with reduced cross-reactivity.

Cellular signal transduction is the term used to describe the mechanism by which all cells (in any organism) transfer information (1). This process involves carefully orchestrated and tightly controlled events that are largely mediated by protein molecules. Proteins vary in size and function but in essence are the ‘work horse’ of the cell. One of the most significant protein ‘work horses’ is the family of proteins termed the protein kinases; enzymes that transfer a phosphate group from the ubiquitous energy molecule ATP to the surfaces of other proteins (2). The action of the protein kinases creates a phosphorylation cascade that is largely responsible for information transfer (3). Phosphorylation of a target protein creates a new function that leads to phosphorylation of another target that continues the flow of information. Ultimately, the nucleus is directed to carry out the message; for example, cell proliferation, cell growth, cell death.

Precise spatial and temporal control of the kinase mediated phosphorylation cascades is absolutely critical to the health of a cell and in turn to the health of the entire organism. Numerous diseases arise from improper regulation of kinase mediated signaling. For example, if a kinase is not regulated and continues to phosphorylate its target in the absence of an extracellular signal, spurious cell growth and proliferation (cancer) can occur (4). By the same token, exerting control over particular kinases can be useful in the treatment of disease. For example, in autoimmune diseases one strategy might be to inhibit the function of a kinase molecule to stop immune cell activation. Likewise, organ transplant patients need their immune systems suppressed to avoid rejection of the transplanted organ (5); one way to achieve this suppression is to inhibit kinase signaling that leads to immune cell activation. Thus, drugs that target kinase molecules have extensive therapeutic applications yet our knowledge of how to develop such drugs is still limited.

The current approach in the pharmaceutical industry for targeting protein kinase activity in cellular signaling pathways is to develop active site inhibitors (6). The active site of an enzyme is the location on the molecular where the chemistry occurs. Thus, identifying small molecules that can ‘clog up’ an active site is an effective way to inhibit kinase function and its corresponding signaling pathway. The problem with this current approach is that most protein kinase active sites look like most other protein kinase active sites (this makes a lot of sense since they all carry out the same chemical reaction). It is therefore very difficult to find small molecules that bind to only one active site and not many kinase active sites. The human genome codes for over 500 protein kinases and so it is very important to develop therapeutics that hit the desired target and not tens of other target as well. Thus, cross-reactivity (and corresponding unwanted side effects) has hindered drug development in this area.

### IL-2 Inducible Tyrosine Kinase (Itk)

Itk is a protein kinase that is present in specialized immune cells called T cells. T cells, or lymphocytes, are white blood cells that play a critical role in cell mediated immunity (7). They are activated when they encounter another type of cell termed the ‘antigen presenting cell’ that carries a piece of a non-host molecule such as a virus. T cell activation is the first step in this cell mediated immunity and Itk is a kinase that acts at the very beginning of the phosphorylation cascade controlling T cell activation. Itk is therefore an excellent target to modulate the immune response prior to full-fledged lymphocyte activation.

My laboratory has been studying the Itk molecule for several years (8-17) and we have very recently gained exciting mechanistic insight into how Itk phosphorylates its targets in T cells. Many protein kinases use their active site to both recognize the target phosphorylation site and carry out the chemistry of phosphorylation. For Itk we have found a different paradigm wherein the target is recognized by a specific interaction between a region outside of the Itk active site and an equally remote site on the target protein. The nature of the protein-protein interaction that occurs during this docking event is novel and not previously described for any protein kinase. The docking interaction is required to achieve target phosphorylation by Itk and interruption of the interaction leads to a loss of target phosphorylation. Thus, the remote docking between Itk and its targets is a prime candidate for modulating Itk function in cells. A small molecule inhibitor of the Itk:target interaction would provide a completely novel means by which this kinase could be controlled in T cells. Quite appealing is the fact that this interaction is well outside of the kinase active site and is to date only characterized for Itk suggesting the intriguing possibility that small molecule therapeutics that interrupt the docking interaction could be extremely specific for this single kinase and not cross-react with other kinases in the cell.

Numerous small molecule libraries exist in facilities that offer high-throughput screens to academic researchers across the country. Two prominent centers are at Harvard University (www,iccb med.harvard.edu/) and Stanford University (http://htbc.stanford.edu). Once a screening methodology for the specific application has been developed by the researcher, staff at these centers are available to assist high-throughput analyses of the more than 150,000 small molecule compounds. The screening process is outlined in FIG. 11 and is designed to identify new tools (small molecules) that can be used to better understand biological processes. As well, small molecules that are identified in this manner serve as lead compounds for future work toward drug development.

The first and most critical step in this process is screen development. The screen must be of sufficient sensitivity, rapid, amenable to high-throughput, reproducible and inexpensive. Our application involves two single target proteins, the Itk kinase itself and the target docking protein (which can be used without its adjacent phosphorylation site to simplify the assay to that of a protein-protein interaction rather than an enzymatic reaction). Our data show that the two proteins interact in a stable fashion and so we will be screening for specific small molecules that break up the interaction.

Fluorescence polarization (FP) provides a means to monitor whether two proteins are associated or disassociated in solution and is completely compatible with high throughput screening technologies (18). FP is a biophysical technique that allows detection of relative molecular motions. We will attach a fluorescent dye to the smaller target protein. Binding of this molecule to the larger kinase molecule will lead to a slower speed of rotation and a concomitant increase in FP of the fluorescent dye compared to the free fluorescently labeled protein. Thus, the kinase:target complex present in each of the 384 wells of the assay plate (FIG. 11) will give a higher FP reading than free target protein. Addition of compound stock solutions to each well will be carried out in an automated fashion and FP will be measured for the individual wells using a plate reader. A decrease in FP (consistent with more rapid speed of rotation of the free fluorescently labeled target protein) will indicate those wells that contain a small molecule capable of disrupting the kinase:target complex. Lead compounds will then be examined more carefully for their properties in disrupting target docking and phosphorylation by the Itk kinase domain.

The Itk kinase domain recognizes its two physiological substrates (an autophosphorylation site on Itk itself and a site on the downstream target PLCγ1) by a direct interaction with a remote substrate-docking site. This serves as a model for a number of SH2 mediated phosphorylation events (FIG. 12). The docking site in the two different substrates is an ‘SH2 domain’; a conserved protein domain that has never previously been assigned the function of recognition module for a protein kinase. The Itk kinase domain and SH2 domain form a specific intermolecular complex that is a prerequisite for target phosphorylation by Itk. We will make use of FP and small molecule library screens to identify lead compounds that disrupt the docking interaction between Itk and the two target SH2 domains. Such compounds will then be characterized for their ability to affect Itk mediated phosphorylation both in vitro and in cells. It should be noted that mouse model systems where the Itk gene has been ‘knocked out’ show significantly reduced T cell mediated immune response (19). Our proposal consists of a series of objectives leading toward discovery and characterization of small molecules that can (in an inducible fashion) eliminate the function of the Itk kinase in T cells thereby reducing T cell mediated immune response. The health related scenarios where this would be therapeutically useful are many and include complete or partial autoimmunity (examples: Lupus erythematosus, Diabetes mellitus, Rheumatoid arthritis, Crohn's disease) and immunosuppression to avoid organ rejection following transplantation.

Objective 1: Chemical modification of each SH2 domain with a specific fluorescent tag.

Objective 2: Measure fluorescence polarization for the SH2 domain alone and bound to Itk kinase domain (or larger fragments of Itk). We expect the difference in measured FP to reflect the size difference between the two states; SH2 is 12 kDa and Itk kinase domain is 32 kDa (that would be the smallest Itk fragment used). Thus, fluorescently labeled SH2 alone will yield a lower FP than the fluorescently labeled SH2 domain bound to Itk or kinase domain containing fragments of Itk (minimally a 44 kDa complex).

Objective 3: Monitor change in FP upon disruption of the Itk:SH2 complex. We have already identified control reagents that will readily disrupt the protein-protein complex. We can thus show that the FP experiment will serve as a screen to identify small molecule reagents that affect the status of the docking interaction.

Objective 4: Screen molecular libraries.

Objective 5: Analyze results of screen and identify specific small molecules to bring forward for biochemical and structural analysis. We have numerous functional assays in the laboratory to probe the effect of small molecules on the Itk docking mechanism. As well, structure determination (by either NMR spectroscopy or x-ray crystallography) of the small molecule bound to its target is well within our area of expertise and will provide valuable insight for the design of second generation compounds.

Objective 6: Test cell permeability of molecules emerging from screening step in Objective 4. Jurkat T cells are excellent models for T cell signaling and our laboratory has experience working with this cell line. Once cell permeability is established effect of small molecules on Itk mediated phopshorylation can be explored.

### Citations

(1) Pawson, T. (2004) Specificity in signal transduction: from phosphotyrosine-SH2 domain interactions to complex cellular systems. Cell 116, 191-203.
(2) Pawson, T. (1994) Tyrosine kinase signaling pathways. Princess Takamatsu Symp 24, 303-22.
(3) Pawson, T., and Scott, J. D. (2005) Protein phosphorylation in signaling—50 years and counting. Trends Biochem Sci 30, 286-90.
(4) Cooper, J. A., and Howell, B. (1993) The when and how of Src regulation. Cell 73, 1051-4.
(5) Schreiber, S. L., and Crabtree, G. R. (1992) The mechanism of action of cyclosporin A and FK506. Immunol Today 13, 136-42.
(6) Liao, J. J. (2007) Molecular recognition of protein kinase binding pockets for design of potent and selective kinase inhibitors. J Med Chem 50, 409-24.
(7) Berg, L. J., Finkelstein, L. D., Lucas, J. A., and Schwartzberg, P. L. (2005) Tec family kinases in T lymphocyte development and function. Annu Rev Immunol 23, 549-600.
(8) Andreotti, A. H. (2003) Native state proline isomerization: an intrinsic molecular switch. Biochemistry 42, 9515-24.
(9) Andreotti, A. H., Bunnell, S. C., Feng, S., Berg, L. J., and Schreiber, S. L. (1997) Regulatory intramolecular association in a tyrosine kinase of the Tec family. Nature 385, 93-7.
(10) Brazin, K. N., Fulton, D. B., and Andreotti, A. H. (2000) A specific intermolecular association between the regulatory domains of a Tec family kinase. J Mol Biol 302, 607-23.
(11) Brazin, K. N., Mallis, R. J., Fulton, D. B., and Andreotti, A. H. (2002) Regulation of the tyrosine kinase Itk by the peptidyl-prolyl isomerase cyclophilin A. Proc Natl Acad Sci USA 99, 1899-904.
(12) Breheny, P. J., Laederach, A., Fulton, D.B. and Andreotti, A.H. (2003) Ligand Specificity Modulated by Prolyl Imide Bond Cis/Trans Isomerization in the Itk SH2 Domain: A Quantitative NMR Study. Journal of the American Chemical Society 125, 15706-15707.
(13) Laederach, A., Cradic, K. W., Brazin, K. N., Zamoon, J., Fulton, D. B., Huang, X. Y., and Andreotti, A. H. (2002) Competing modes of self-association in the regulatory domains of Bruton's tyrosine kinase: intramolecular contact versus asymmetric homodimerization. Protein Sci 11, 36-45.
(14) Laederach, A., Cradic, K. W., Fulton, D. B., and Andreotti, A. H. (2003) Determinants of intra versus intermolecular self-association within the regulatory domains of Rlk and Itk. J Mol Biol 329, 1011-20.
(15) Mallis, R. J., Brazin, K. N., Fulton, D. B., and Andreotti, A. H. (2002) Structural characterization of a proline-driven conformational switch within the Itk SH2 domain. Nat Struct Biol 9, 900-5.
(16) Min, L., Fulton, D. B., and Andreotti, A. H. (2005) A case study of proline isomerization in cell signaling. Front Biosci 10, 385-97.
(17) Pletneva, E. V., Sundd, M., Fulton, D. B., and Andreotti, A. H. (2006) Molecular details of Itk activation by prolyl isomerization and phospholigand binding: the NMR structure of the Itk SH2 domain bound to a phosphopeptide. J Mol Biol 357, 550-61.
(18) Trinquet, E., and Mathis, G. (2006) Fluorescence technologies for the investigation of chemical libraries. Mol Biosyst 2, 380-7.
(19) Liao, X. C., and Littman, D. R. (1995) Altered T cell receptor signaling and disrupted T cell development in mice lacking Itk. Immunity 3, 757-69.

What is claimed is:

1. A drug screening method for identifying agents which will modulate CTK effects for treatment of diseases or conditions associated with the CTK signaling cascade comprising:
screening said agent for its ability to modulate the CTK substrate interaction wherein the interaction involves a site other than the CTK active site.

2. The method of claim 1 wherein said agent inhibits CTK mediated phosphorylation.

3. The method of claim 1 wherein said agent inhibits the docking interaction between CTK and a substrate SH2 domain.

4. The method of claim 1 wherein said agent is a compound selected from the group consisting of: a small molecule, an antibody, a nucleic acid, a carbohydrate, or a protein.

5. The method of claim 3 in which the CTK is from a CTK family selected from the group consisting of: Abl, Csk, FAK, JAK, Src, Syk, and Tec.

6. The method of claim 5 wherein the CTK family is the Src subfamily and includes a protein kinase selected from the group consisting of: Src, Fyn, Hck, Lyn, Lck Yes, Yrk, Blk, Fgr.

7. The method of claim 6 wherein said Src subfamily protein kinase includes one or more of the CTK/substrate combinations:
Lck/Itk; Lyn/Btk or Tec; Fyn/Rlk; and Src/Bmx.

8. The method of claim 5 wherein the CTK is from the Tec sub family.

9. The method of claim 8 wherein said CTK is selected from the group consisting of:
Itk, Btk, Tec, Bmx, and Rlk.

10. The method of claim 9 wherein said CTK/substrate combination includes one or more of the following: Itk/PLCγ1 or Itk or PLCγ2; Btk/PLCγ2 or Btk, Tec/ PLCγ1 or Tec or PLCγ2; Rlk/ PLCγ1 or Rlk or PLCγ2; and Bmx/Bmx or PLCγ2 or Stat3.

11. A method for treating cancer, psoriasis, hepatic cirrhosis, diabetes, atherosclerosis, angiogenesis, restenosis, ocular diseases, rheumatoid arthritis and other inflammatory disorders, autoimmune conditions, and immunosuppression associated with CTK regulation comprising:
administering a compound that modulates a protein—protein interaction of a nonreceptor protein tyrosine kinase (CTK) and a substrate SH2 domain wherein said interaction does not involve the active site of the kinase.

12. The method of claim 11 wherein said agent inhibits CTK mediated phosphorylation.

13. The method of claim 11 wherein said compound inhibits the docking interaction between CTK and a substrate SH2 domain.

14. The method of claim 11 wherein said compound is selected from the group consisting of: a small molecule, an antibody, a nucleic acid, a carbohydrate, or a protein.

15. The method of claim 11 in which the CTK is from a CTK family selected from the group consisting of: Abl, Csk, FAK, JAK, Src, Syk, and Tec.

16. The method of claim 15 wherein the CTK family is the Src subfamily and includes a protein kinase selected form the group consisting of: Src, Fyn, Hck, Lyn, Lck Yes, Yrk, Blk, Fgr.

17. The method of claim 16 wherein said Src subfamily protein kinase includes one of more of the CTK/substrate combinations:
Lck/Itk; Lyn/Btk or Tec; Fyn/Rlk; and Src/Bmx.

18. The method of claim 15 wherein the CTK is from the Tec sub family.

19. The method of claim 18 wherein said CTK is selected from the group consisting of:
Itk, Btk, Tec, Bmx, and Rlk.

20. The method of claim 19 wherein said CTK/substrate combinations includes one or more of the following: Itk/PLCγ1 or Itk or PLCγ2; Btk/PLCγ2 or Btk, Tec/PLCγ1 or Tec or PLCγ2; Rlk/PLCγ1 or Rlk or PLCγ2; and Bmx/Bmx or PLCγ2 or Stat3.

21. A pharmaceutical composition for the treatment of a CTK related disorder comprising: a compound that inhibits activation of a CTK cascade by inhibiting the substrate SH2/CTK protein interaction and a pharmaceutically acceptable carrier.

22. The pharmaceutical composition of claim 21 in which the CTK is from a CTK family selected from the group consisting of: Abl, Csk, FAK, JAK, Src, Syk, and Tec.

23. The pharmaceutical composition of claim 22 wherein the CTK family is the Src subfamily and includes a protein kinase selected form the group consisting of: Src, Fyn, Hck, Lyn, Lck Yes, Yrk, Blk, Fgr.

24. The pharmaceutical composition of claim 23 wherein said Src subfamily protein kinase includes one of more of the CTK/substrate combinations:
Lck/Itk; Lyn/Btk or Tec; Fyn/Rlk; and Src/Bmx.

25. The pharmaceutical composition of claim 22 wherein the CTK is from the Tec sub family.

26. The pharmaceutical composition of claim 25 wherein said CTK is selected from the group consisting of: Itk, Btk, Tec, Bmx, and Rlk.

27. The pharmaceutical composition of claim 26 wherein said CTK/substrate combinations includes one or more of the following: Itk/PLCγ1 or Itk or PLCγ2; Btk/PLCγ2 or Btk, Tec/PLCγ1 or Tec or PLCγ2; Rlk/PLCγ1 or Rlk or PLCγ2; and Bmx/Bmx or PLCγ2 Stat3.

28. The pharmaceutical composition of claim 21 wherein said agent inhibits CTK mediated phosphorylation.

29. The pharmaceutical composition of claim 21 wherein said compound inhibits the docking interaction between CTK and a substrate SH2 domain.

30. The pharmaceutical composition of claim 21 wherein said compound is selected from the group consisting of: a small molecule, an antibody, a nucleic acid, a carbohydrate, or a protein.

31. A method for screening for a pharmaceutical composition for treating a CTK related disorder comprising:
(a) administering the test compound to a cell expressing CTK;
(b) measuring the enzymatic activity or downstream cellular consequences of the enzymatic activity (e.g. cytokine production) of a component of the CTK signaling pathway; and
(c) determining whether the test compound increases or decreases the level of enzymatic activity of the component of the CTK signaling pathway, by interacting with the SH2 domain, in which test compounds that result in increased or decreased enzymatic activity of the component of the CTK signaling pathway is identified as an agent which modulates the CTK.

32. The method of claim 31 in which the CTK is from a CTK family selected from the group consisting of: Abl, Csk, FAK, JAK, Src, Syk, and Tec.

33. The method of claim 31 wherein the CTK family is the Src subfamily and includes a protein kinase selected form the group consisting of: Src, Fyn, Hck, Lyn, Lck Yes, Yrk, Blk, Fgr.

34. The method of claim 33 wherein said Src subfamily protein kinase includes one of more of the CTK/substrate combinations:
Lck/Itk; Lyn/Btk or Tec; Fyn/Rlk; and Src/Bmx.

35. The method of claim 32 wherein the CTK is from the Tec sub family.

36. The method of claim 35 wherein said CTK is selected from the group consisting of:
Itk, Btk, Tec, Bmx, and Rlk.

37. The method of claim 36 wherein said CTK/substrate combinations includes one or more of the following: Itk/PLCγ1 or Itk or PLCγ2; Btk/PLCγ2 or Btk, Tec/ PLCγ1 or Tec or PLCγ2; Rlk/ PLCγ1 or Rlk or PLCγ2; and Bmx/Bmx or PLCγ2 or Stat3.

38. The method of claim 31 wherein said agent inhibits CTK mediated phosphorylation.

39. The method of claim 31 wherein said compound inhibits the docking interaction between CTK and a substrate SH2 domain.

40. The method of claim 31 wherein said compound is selected from the group consisting of: a small molecule, an antibody, a nucleic acid, a carbohydrate, or a protein.

41. A method for screening for a pharmaceutical composition for treating a CTK related disorder comprising:
(a) administering the test compound to a cell expressing CTK;
(b) measuring the binding between the CTK enzyme and its substrate at an SH2 domain; and
(c) determining whether the test compound increases or decreases the association of the enzyme and an SH2 domain, in which test compounds that result in increased or decreased association at the SH2 site is identified as an agent which modulates the CTK.

42. The pharmaceutical composition of claim 41 in which the CTK is from a CTK family selected from the group consisting of: Abl, Csk, FAK, JAK, Src, Syk, and Tec.

43. The method of claim 42 wherein the CTK family is the Src subfamily and includes a protein kinase selected form the group consisting of: Src, Fyn, Hck, Lyn, Lck Yes, Yrk, Blk, Fgr.

44. The method of claim 43 wherein said Src subfamily protein kinase includes one of more of the CTK/substrate combinations:
Lck/Itk; Lyn/Btk or Tec; Fyn/Rlk; and Src Bmx.

45. The method of claim 42 wherein the CTK is from the Tec sub family.

46. The method of claim 45 wherein said CTK is selected from the group consisting of:
Itk, Btk, Tec, Bmx, and Rlk.

47. The method of claim 46 wherein said CTK/substrate combinations includes one or more of the following: Itk/PLCγ1 or Itk or PLCγ2; Btk/PLCγ2 or Btk, Tec/ PLCγ1 or Tec or PLCγ2; Rlk/ PLCγ1 or Rlk or PLCγ2; and Bmx/Bmx or PLCγ2 or Stat3.

48. The method of claim 41 wherein said agent inhibits CTK mediated phosphorylation.

49. The method of claim 41 wherein said compound inhibits the docking interaction between CTK and a substrate SH2 domain.

50. The method of claim 41 wherein said compound is selected from the group consisting of: a small molecule, an antibody, a nucleic acid, a carbohydrate, or a protein.

51. A method for modulating the CTK pathway for treatment of a CTK related disorder comprising:
administering a compound which includes an SH2 domain to cells with a CTK enzyme.

52. The method of claim 51 wherein said exogenous SH2 domain is added to a CTK substrate and said SH2 increases CTK mediated phosphorylation.

53. The method of claim 52 wherein said exogenous SH2 domain is not part of a CTK substrate and competitively inhibits CTK interacting with its substrate.
